(19) United States
(12) Patent Application Publication
(10) Pub. No.: US 2015/0273010 A1
(43) Pub. Date: Oct. 1, 2015

(54) HIGH-THROUGHPUT ASSAYS FOR HIV-1 ENVELOPE MATURATION AND NOVEL PEPTIDE INHIBITORS AGAINST HIV-1

(71) Applicant: SAN DIEGO STATE UNIVERSITY (SDSU) FOUNDATION, San Diego, CA (US)

(72) Inventors: Roland Wolkowicz, San Diego, CA (US); Zachary Stolp, San Diego, CA (US); Samantha Diaz, San Diego, CA (US)

(21) Appl. No.: 14/434,622

(22) PCT Filed: Oct. 9, 2013
(86) PCT No.: PCT/US13/64077

§ 371 Date: Apr. 9, 2015

Related U.S. Application Data
(60) Provisional application No. 61/711,637, filed on Oct. 9, 2012.

(51) Int. Cl. A61K 38/08; A61K 45/06; C12Q 1/37; C07K 7/06

(52) U.S. Cl. CPC A61K 38/08; C07K 7/06; A61K 45/06; C12Q 1/37; C07K 2319/33; G01N 2500/04; G01N 2500/10; G01N 2333/9513

(57) ABSTRACT

The invention provides peptide inhibitors against human immunodeficiency virus (HIV), including HIV-1, and compositions, formulations, and pharmaceutical compositions comprising same, and methods of making and using them. The invention provides methods for treating, ameliorating, slowing the progress of, reversing, or preventing a retroviral infection, an HIV infection, or an HIV-1 infection, or AIDS. In alternative embodiments, the invention provides compositions, including pharmaceutical compositions and formulations, comprising peptides or polypeptide having a sequence as set forth in SEQ ID NO:1 and/or SEQ ID NO:2, and peptidomimetics and bioisosteres thereof. In alternative embodiments, the invention provides cell-based platforms, multiplexed systems or platforms, or cell-based methods, for monitoring the activity of an HIV-1 protease (PR) enzyme, or for screening for a substrate of the HIV-1 protease (PR) enzyme.

### TECHNICAL FIELD

This invention relates to molecular and cellular biology, biochemistry, molecular genetics, and drug design and discovery. The invention provides peptide inhibitors against human immunodeficiency virus (HIV), including HIV-1, and compositions, formulations, and pharmaceutical compositions comprising same, and methods of making and using them. The invention provides methods for treating, ameliorating, slowing the progress of, reversing, or preventing a retroviral infection, an HIV infection, or an HIV-1 infection, or AIDS. In alternative embodiments, the invention provides cell-based platforms, multiplexed systems or platforms, or cell-based methods, for monitoring the activity of an HIV-1 protease (PR) enzyme, or for screening for a substrate of the HIV-1 protease (PR) enzyme.

### BACKGROUND

Viral infection is intrinsically the result, at the molecular level, of viral proteins interacting with their cellular counterparts, defining the nature of the virus/host crosstalk. Many protein/protein interaction studies have described the viral/viral and viral/host protein interactions crucial for the establishment of viral infection, but the knowledge about the nature of their interactions is far from complete. The intricacies of virus/host crosstalk are exemplified by proteolytic enzymes from both viral and host origin. Viruses are known to utilize the host proteolytic machinery for the cleavage of their own proteome. The case of Ebola virus (Filoviridae) glycoprotein, cleaved by cellular cathepsin L to facilitate receptor binding or the avian infectious bursal disease virus (Birnaviridae) VP2 capsid precursor protein, cleaved by puromycin-sensitive aminopeptidase host protease for viral particle maturation, are just two examples. On the other hand, viral proteases, which are essential for the production of infectious viral particles, have been found to cleave cellular substrates as well.

For human immunodeficiency virus-1 (HIV-1), protease (PR, or HIV-1 protease) is responsible for the post-translational cleavage of the HIV Gag and Gag-Pol poly-protein precursor proteins, in the host, cleaving all sites of the viral proteome but that of envelope, which is cleaved by furin and other host protein convertases in the endoplasmic reticulum. PR also has autocatalytic properties, enabling its own removal from the precursor poly-protein, following dimerization of the Gag-Pol precursor polyprotein. The HCV NS3/4A protease cleaves all non-structural proteins while structural proteins are cleaved by the host. For other Flaviviridae family, viruses such as Dengue and West Nile viruses, a very similar picture emerges. The viral NS3 protease (NS3pro) is required for the complete processing of the polyprotein precursor and its transformation into mature viral proteins where NS3pro cleaves the structural protein capsid and the boundaries between the non-structural proteins and host proteases furin and secretase cleave the rest.

While viral protease recognition/cleavage sites have been identified within the host proteome, few if any assays have been developed to identify those targets in a cellular milieu.

### SUMMARY

In alternative embodiments, the invention provides isolated, synthetic or recombinant polypeptides or peptides comprising or consisting of:

(a) an amino acid sequence comprising or consisting of the formula:

(SEQ ID NO: 1)
Thr Arg Arg Val Ala His Asn Ser Glu (TRRVAHNSE);
or
(SEQ ID NO: 2)
Trp Arg Gly Ala Ala Met Val Arg Gly (WRGAAMVRG);

(b) a peptidomimetic or a bioisostere of the isolated, synthetic or recombinant polypeptide or peptide of (a); or

(c) the polypeptide or peptide of (a), or the peptidomimetic or bioisostere of (b), further comprising, or modified by: acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation and/or arginylation.

In alternative embodiments, the invention provides chimeric proteins or peptides comprising an isolated, synthetic or recombinant polypeptide or peptide of the invention (e.g., SEQ ID NO:1, or SEQ ID NO:2, or peptidomimetics or bioisosteres thereof), wherein the chimeric protein comprises:

(A)

(a) (i) a first domain comprising an isolated, synthetic or recombinant polypeptide or peptide of the invention (e.g., SEQ ID NO:1, or SEQ ID NO:2, or peptidomimetics or bioisosteres thereof); and, (ii) at least a second domain or moiety;

(b) the chimeric protein of (a), wherein the chimeric protein comprises a recombinant fusion protein; or

(c) the chimeric protein of (a) or (b), wherein the second domain or moiety comprises a targeting agent;

(B) the chimeric protein of (A), wherein the targeting agent comprises an antibody Fc domain or an antibody that binds to an Fc receptor, or a chimeric protein comprising two or more antibody Fc domains;

(C) the chimeric protein of (A) or (B), wherein the at least second domain or moiety comprises an Fc domain, a protein C, an antibacterial or bacteriostatic peptide or protein, an antibiotic, a cytokine, an immunoregulatory agent, an anti-inflammatory agent, a complement activating agent, a carbohydrate-binding domain or a combination thereof;

(D) the chimeric protein of any of (A) to (C), wherein the chimeric protein comprises a recombinant, peptidomimetic or synthetic protein; or

(E) the chimeric protein of any of (A) to (D), wherein the first domain is joined to the second domain or moiety by a chemical linking agent.

In alternative embodiments, the invention provides compositions comprising:

(a) a first composition comprising the isolated, synthetic or recombinant polypeptide or peptide of the invention, or the chimeric protein of the invention; and a second composition;

(b) the composition of (a), wherein the second composition comprises a liquid, a lipid or a powder;

(c) the composition of (a) or (b) formulated as a protein preparation, wherein optionally the protein preparation comprises a liquid, a slurry, a powder, a spray, a suspension, a lyophilized composition/formulation, a solid, geltab, pill, implant, a gel; or a pharmaceutical formulation, a food or a feed or a supplement thereof; or

(d) the composition of (a) or (b) immobilized on or inside a cell, a vesicle, a liposome, a film, a membrane, a metal, a resin, a polymer, a ceramic, a glass, a microelectrode, a graphitic particle, a bead, a gel, a plate, an array, a capillary tube, a crystal, a tablet, a pill, a capsule, a powder, an agglomerate, a surface, or a porous structure.

In alternative embodiments, the invention provides liposomes comprising: (a) the isolated, synthetic or recombinant polypeptide or peptide of the invention, or the chimeric protein of the invention; or, (b) the liposome of (a), wherein the liposome is formulated with a pharmaceutically acceptable excipient.

In alternative embodiments, the invention provides pharmaceutical compositions comprising: an isolated, synthetic or recombinant polypeptide or peptide of the invention, or a chimeric protein of the invention, or a composition of the invention, or a liposome of the invention, or any combination thereof; and, a pharmaceutically acceptable excipient.

In alternative embodiments, the invention provides inhalants or spray formulations comprising: an isolated, synthetic or recombinant polypeptide or peptide of the invention, or a chimeric protein of the invention, or a composition of the invention, a liposome of the invention, or a pharmaceutical composition of the invention, or any combination thereof; and, a pharmaceutically acceptable excipient.

In alternative embodiments, the invention provides parenteral formulations comprising: an isolated, synthetic or recombinant polypeptide or peptide of the invention, or a chimeric protein of the invention, or a composition of the invention, a liposome of the invention, or a pharmaceutical composition of the invention, or any combination thereof; and, a pharmaceutically acceptable excipient.

In alternative embodiments, the invention provides enteral formulations comprising: an isolated, synthetic or recombinant polypeptide or peptide of the invention, or a chimeric protein of the invention, or a composition of the invention, a liposome of the invention, or a pharmaceutical composition of the invention, or any combination thereof; and, a pharmaceutically acceptable excipient.

In alternative embodiments, the invention provides isolated, synthetic or recombinant nucleic acids encoding the isolated, synthetic or recombinant polypeptide or peptide of the invention, or the chimeric protein of the invention.

In alternative embodiments, the invention provides an expression cassette, vector, cloning vehicle, expression vector, cloning vector comprising the nucleic acid of the invention, or having contained therein a nucleic acid of the invention. In alternative embodiments, the cloning vehicle comprises or is a viral vector, a plasmid, a phage, a phagemid, a cosmid, a fosmid, a bacteriophage or an artificial chromosome, and optionally the viral vector comprises an adenovirus vector, a retroviral vectors or an adeno-associated viral vector, and optionally the expression cassette, vector, cloning vehicle, expression vector, cloning vector comprises or is contained in a bacterial artificial chromosome (BAC), a plasmid, a bacteriophage P1-derived vector (PAC), a yeast artificial chromosome (YAC), a mammalian artificial chromosome (MAC).

In alternative embodiments, the invention provides a cell, a transformed cell or a host cell comprising: the nucleic acid of the invention, or the expression cassette, vector, cloning vehicle, expression vector, cloning vector of the invention. The cell can be a bacterial cell, a mammalian cell, a fungal cell, a yeast cell, an insect cell or a plant cell; and optionally the bacterial cell is any species within the genera Escherichia, Bacillus, Streptomyces, Salmonella, Pseudomonas or Staphylococcus, or Escherichia coli, Lactococcus lactis, Bacillus subtilis, Bacillus cereus, Salmonella typhimurium or Pseudomonas fluorescens.

In alternative embodiments, the invention provides methods for treating, ameliorating, slowing the progress of, reversing, or preventing a retroviral infection, or an HIV-1 infection, or AIDS, in an individual in need thereof, comprising:

(A) (a) providing the isolated, synthetic or recombinant polypeptide or peptide of the invention (e.g., SEQ ID NO:1, or SEQ ID NO:2, or peptidomimetics or bioisosteres thereof), the chimeric protein of the invention, the composition of the invention, the liposome of the invention, the pharmaceutical composition of the invention, the inhalant or spray formulation of the invention, the parenteral formulation of the invention, or the enteral formulation of the invention; and

(b) administering an effective amount of (a) to the individual, thereby treating, ameliorating, slowing the progress of, reversing, or preventing a retroviral infection, or an HIV-1 infection, or AIDS; or,

(B) the method of (A), further comprising co-administration of at least one anti-retroviral or anti-HIV-1 drug, composition, therapy or diet.

In alternative embodiments, the invention provides an isolated, recombinant or synthetic nucleic acid comprising or consisting of a construct as set forth in FIG. 2, FIG. 6, FIG. 7, FIG. 9B or FIG. 10B.

In alternative embodiments, the invention provides a vector, expression cassette, cosmid or plasmid comprising or having contained therein the isolated, recombinant or synthetic nucleic acid of the invention.

In alternative embodiments, the invention provides a cell, a transformed cell or a host cell comprising, or having contained therein, the isolated, recombinant or synthetic nucleic acid of the invention, or the vector, expression cassette, cosmid or plasmid of the invention.

In alternative embodiments, the invention provides a chimeric polypeptide encoded by a nucleic acid comprising or consisting of a construct as set forth in FIG. 2, FIG. 6, FIG. 7, FIG. 9B or FIG. 10B.

In alternative embodiments, the invention provides a cell, a transformed cell or a host cell comprising, or having contained therein, the chimeric polypeptide of the invention.

In alternative embodiments, the invention provides a chimeric or hybrid polypeptide comprising (or consisting of): (a) the polypeptide encoded by the nucleic acid of the invention; or (b) the chimeric (hybrid) protein of (a), wherein the protein comprises a synthetic protein or peptide, recombinant protein or peptide, a peptidomimetic or a combination thereof.

In alternative embodiments, the invention provides a cell, a transformed cell or a host cell comprising the isolated, recombinant or synthetic nucleic acid of the invention, or the vector, expression cassette, cosmid or plasmid of the invention, wherein optionally the cell is a mammalian or a human cell, wherein optionally the cell is a hepatocyte, or a lymphocyte or a T cell, or a CD4+ T cell.

In alternative embodiments, the invention provides cell-based platforms, a multiplexed system or platform, or a cell-based method, for monitoring the activity of an enzyme, or an HIV-1 protease (PR) enzyme, or for screening for a substrate of the enzyme or, the HIV-1 protease (PR) enzyme, comprising:

(1) (a) providing:
(i) a construct of the invention, or a vector, expression cassette, cosmid or plasmid of the invention, or a nucleic acid encoding the chimeric (hybrid) protein of the invention; or
(ii) a pairing or grouping of constructs as set forth in FIG. 1 or FIG. 6; or
(iii) a pairing or grouping of constructs comprising:
   (1) a construct wherein an enzyme, or a PR HIV-1, is fused between a DNA-binding domain (DBD) and a transactivation domain (TAD) of a transcription factor (optionally a Gal4 transcription factor), wherein the enzyme or PR fused within the transcription factor auto-catalytically can cleave itself resulting in two non-functional transcription factor domains, and
   (2) a construct encoding a detectable moiety or protein operatively linked to a transcription factor (optionally a promoter) which is only active when activated by the transactivation domain (TAD) of a transcription factor (optionally a Gal4 transcription factor) of (1),
   and optionally the detectable moiety or protein comprises a fluorescent protein, a green fluorescent protein (GFP), a far-red fluorescent protein, an E2 crimson fluorescent protein, or equivalents thereof;

and, a cell comprising an environment capable of supporting the expression of the chimeric (hybrid) protein by the nucleic acid;

(b) inserting or transfecting or infecting the nucleic acid, vector, recombinant virus, cloning vehicle, expression cassette, cosmid or plasmid of (a) into the cell; and

(c) contacting the cell with a putative (test) enzyme inhibitor or activator,

wherein optionally the enzyme (optionally an HIV-1 protease (PR) enzyme) is provided in cis or in trans,

wherein optionally an enzyme (optionally HIV-1 protease (PR) enzyme) inhibitor or activator is added to the cell before, during and/or after inserting (transfecting) the nucleic acid, vector, recombinant virus, cloning vehicle, expression cassette, cosmid or plasmid of (a) into the cell and/or expressing the chimeric protein encoded by a nucleic acid of (a) in the cell,

and optionally the cell-based method further comprises a negative, positive and/or alternative control set of cells into which the nucleic acid, vector, recombinant virus, cloning vehicle, expression cassette, cosmid or plasmid of (a) also has been inserted (or transfected) and expresses the chimeric protein encoded by a nucleic acid of (a), but the negative control set of cells is not exposed to an enzyme (optionally an HIV-1 protease (PR) enzyme) inhibitor or activator, or is exposed to a different or known enzyme substrate (optionally an HIV-1 protease (PR) enzyme substrate), or a different amount of substrate or inhibitor or activator, or a positive control wherein the cells are exposed to a known substrate of the enzyme; and

(d) determining whether a putative (test) enzyme substrate is sufficiently cleaved by the enzyme or an enzymatically active fragment thereof,

optionally by measuring the ability of the enzyme inhibitor to partially or completely inhibit cleavage of the putative (test) enzyme substrate; or

determining whether the putative inhibitor of the enzyme inhibits the enzyme to result in sufficient active transcription factor (optionally Gal4 transcription factor) to be produced to activate production of the detectable moiety or protein, which optionally comprises a fluorescent protein, a green fluorescent protein (GFP), a far-red fluorescent protein, an E2 crimson fluorescent protein, or equivalents thereof (if the enzyme is inhibited partially or completely, it does not cleave the transcription factor, which can then activate production of the detectable moiety or protein),

wherein optionally the fluorescent protein is detected by a fluorescent activated cell sorter (FACS);

(2) the cell-based platform, multiplexed system or platform, or cell-based method of (1), further comprising measuring the ability of the enzyme (optionally a protease, or a HIV-1 protease (PR) enzyme), to partially or completely cleave the enzyme substrate comprises detecting and/or measuring the amount of tag or detection moiety, or “scaffold”, on the cell surface;

(3) the cell-based platform, multiplexed system or platform, or cell-based method of (1) or (2), wherein the cell is a hepatocyte, a lymphocyte or a T cell, or a CD4+ T cell, or a human cell;

(4) the cell-based platform, multiplexed system or platform, or cell-based method of any of (1) to (3), further comprising running a negative control comprising dividing the plurality of the cells co-expressing a nucleic acid of (a) in the cell and not adding the substrate to be screened (the putative (test) substrate) to one of the divided cell samples;

(5) the cell-based platform, multiplexed system or platform, or cell-based method of any of (1) to (4), further comprising running a positive control comprising dividing the plurality of the cells co-expressing the nucleic acid of (a) in the cell and adding a known substrate of the enzyme, or a known substrate of a furin enzyme, a calcium-dependent protein convertase enzyme, prohormone convertase-1 (PC1) enzyme, or an enzyme from a member of the subtilisin/kexin family of proprotein convertases, to one of the divided cell samples; or

(6) the cell-based platform, multiplexed system or platform, or cell-based method of any of (1) to (5), wherein:
(a) the transcriptional regulatory unit comprises a promoter, an inducible promoter or a constitutive promoter;
(b) the cell is a mammalian cell, a monkey cell or a human cell, or the cell is a mammalian cell, a monkey cell or a human cell, or a hepatocyte, a lymphocyte, or a T cell, or a CD4- or CD8-expressing cell, and optionally the cells are genetically bar-coded;
(c) the detectable moiety or protein, which optionally comprises a fluorescent protein, a green fluorescent protein (GFP), a far-red fluorescent protein, an E2 crimson fluorescent protein, or equivalents thereof; or the tag or detection moiety, or “scaffold”; is detected or measured in the cell or on the cell surface by a high throughput screen, a plate reader, a flow cytometry or a microscope visualization;
(d) the (putative) inhibitor of the enzyme comprises a small molecule, a nucleic acid, a polypeptide or peptide, a peptidomimetic, a polysaccharide and/or a lipid; or
(e) the substrate to be screened is a member of a library of substrates to be screened, or a member of a random peptide or polypeptide library; or

(6) the cell-based platform, multiplexed platform or system, or cell-based method of any of (1) to (6), wherein two or more, or a plurality of, enzymes or proteases are screened in the same cell, wherein optionally they are variants of the same enzyme or protease, or different enzymes or proteases, or a combination thereof.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 schematically illustrates an exemplary assay of the invention, as described in detail in Example 2, below: FIG. 1A, Wild type Gal4 as control, no Dox; Without Dox, rtTA cannot bind to the Tet-responsive element (TRE) and induce Gal4 expression, consequently, no GFP expression is expected from the reporter construct; FIG. 1B, In the presence of Dox (the “D” in the grey box), Gal4 expression is induced, activating GFP expression upon binding of rtTA to the 5×UAS element of the reporter construct; FIG. 1C, The Gal4/PR fusion-based system, In the presence of Dox, Gal4/PR is expressed; however, its catalytic activity results in the separation of the Gal4 domains, and thus no GFP expression; FIG. 1D, Same scenario as in C but in the presence of PI (orange circles).

FIG. 2 schematically illustrates exemplary vectors used to practice the invention, as described in detail in Example 2, below: including: a retroviral rtTA expression vector (p-rtTA); a lentiviral reporter vector with the UAS Gal4 responsive element (five copies) upstream of a minimal Cytomegalovirus promoter driving GFP expression (pUAS-GFP); the inducible Gal4-based lentiviral vectors; control (pTRE-Gal4); mutant (D25A) and wild-type PR fusions (pTRE-Gal4/PRm and pTRE-Gal4/PR).

FIG. 3 schematically and graphically illustrates data corroborating that insertion of a PR cleavable domain between a DBD and TAD of Gal4 does not disrupt its transcriptional activity, as described in detail in Example 2, below, Example 3A schematically illustrates a FACS assay/flow cytometry analysis output and FIG. 3B, graphically summarizes this data, as described in detail in Example 2.

FIG. 4 schematically illustrates the results of transient experiments in 293T cells having exemplary vectors of the invention, with PR supplied in trans: flow cytometry analysis of cells transfected with Gal4-RT/IN substrate fusion and GFP reporter (left panel, in the presence of PR (mid panel) or PR and PI (right panel); as described in detail in Example 2, below.

FIG. 5 illustrates a biochemical Assay for PR activity: FIG. 5A illustrates a Western blot where HEK 293T cells were transfected with p96ZM651gag-opt (lanes 1 and 2) and pcDNA empty vector (lanes 3 and 4) or wild-type PR (lanes 5 and 6) or the inactive PR mutant (lanes 7 and 8) in the presence or absence of PI, and transfected cells were analyzed 48 hours post-transfection with anti-p24 antibody, and the gag precursor (p55) and the end product (p24) are shown along with the intermediate products (p42 and p33); and FIG. 5B graphically illustrates a quantification of these Western blots; as described in detail in Example 2, below.

FIG. 6 schematically illustrates exemplary assays of the invention; as described in detail in Example 2, below:

FIG. 6A schematically illustrates an exemplary assay where PR is supplied in trans: FIG. 6A, wild type Gal4 as control. E2-C expression is expected from the reporter construct; FIG. 6B, in the presence of Dox (the “D” grey box), PR expression is induced, and in the presence of a cleavable site, the Gal4 domains are separated, and thus no E2-C expression is expected; FIG. 6C, as in B but in the absence of a cleavable site, Gal4 domains remain intact, resulting in the induction of E2-C expression; FIG. 6D, as in FIG. 6B but in the presence of PI (orange circles), Gal4 fusion remains intact, resulting in the induction of E2-C expression, as described in detail in Example 2, below.

FIG. 6B schematically illustrates an exemplary assay where PR supplied in cis: FIG. 6A, wild type Gal4 as control, in the presence of Dox (the “D” grey box), E2-C expression is expected from the reporter construct; FIG. 6B, PR fusion expression is induced, in the presence of a cleavable site, the Gal4 domains are separated, and thus no E2-C expression is expected; FIG. 6C, as in FIG. 6B, but in the absence of a cleavable site, Gal4 domains remain intact, resulting in the induction of E2-C expression; FIG. 6D, as in FIG. 6B but in the presence of a PI (orange circles), Gal4 fusion remains intact, resulting in the induction of E2-C expression; as described in detail in Example 2, below.

FIG. 7 schematically illustrates exemplary vectors used to practice the invention, as described in detail in Example 2, below, including retroviral/lentiviral constructs utilized in exemplary assays of the invention, where constructs are utilized to generate infectious particles for the transduction of SupT1 cells with the various assay elements, including the Gal4-based vectors: Gal4/Lib and PR for scenario #1 and Gal/Lib/PR for scenario #2, and rtTA, and E2-Crimson reporter constructs, as described in detail in Example 2, below.

FIG. 8A illustrates an outline for constructing a library to practice this invention; and FIG. 8B illustrates the calculation of the percentage of the functional library within the overall library complexity, as described in detail in Example 2, below.

FIG. 9 illustrates an exemplary cell sorting/FACS selection process and screening for an embodiment (Scenario #1) where PR is supplied in trans in an inducible manner and before induction, cells expressing the Gal4/library fusion and thus E2-Crimson-positive, FIG. 9A(a), are sorted out, as illustrated in FIG. 9A(b); following Dox induction, E2-Crimson negative cells, FIG. 9A(c), are sorted out, as illustrated in FIG. 9A(d), and corroborated with PR inhibitor (PI), as illustrated in FIG. 9A(e); the inducible PR construct used in this exemplary cell sorting/FACS selection process is illustrated in FIG. 9B; as described in detail in Example 2, below.

FIG. 10 illustrates the exemplary selection process and screening assay embodiment of the invention (Scenario #2), where PR is supplied in cis as part of the Gal4-fusion; and following Dox induction, E2-Crimson negative cells, as illustrated in FIG. 10A(a) are sorted out, as illustrated in FIG. 10A (b); and following addition of Dox and PI, E2-Crimson positive cells, as illustrated in FIG. 10A(c), are sorted out, as illustrated in FIG. 10A(d); as described in detail in Example 2, below. The inducible PR construct used in this exemplary cell sorting/FACS selection process is illustrated in FIG. 10B; as described in detail in Example 2, below.

As illustrated in FIG. 10C, whether clones are obtained from the negative screen described in the exemplary embodiment Scenario #1 (see FIG. 9A, 9B), or the positive screen described in the embodiment Scenario #2 (see FIG. 10A, 10B), only those that behave as expected are analyzed further, and to corroborate their expected behavior, they can be left untreated for a period of one to two weeks and then treated; as described in detail in Example 2, below.

FIG. 11 illustrates the results of a FACS/flow cytometry analysis, a transient experiment with HEK 293T cells; in particular, a flow cytometry analysis of cells transfected with E2-C reporter and rtTA alone (negative control), or in addition to PR/RT (upper panels) or RT/IN (lower panels) substrate, substrate with PR, or substrate with PR and Darunavir as PI; as described in detail in Example 2, below.

FIG. 12 illustrates results of the transfection of PR with HEK 293T cells stably expressing rtTA and cellular substrates: FIG. 12A, five hours following transfection with mCherry/PR, Darunavir as PI was added; five hours later, Dox was added to activate Gal4/vimentin or Gal4/eIF3d substrate expression; and, FIG. 12B illustrates the same assay as FIG. 12A for Gal4/vimentin but with Gag-PR as source of PR; as described in detail in Example 2, below.

Like reference symbols in the various drawings indicate like elements.

### DETAILED DESCRIPTION

### Peptide Inhibitors Against HIV-1

The invention provides peptide inhibitors against human immunodeficiency virus (HIV), including HIV-1, and compositions, formulations, and pharmaceutical compositions comprising same, and methods of making and using them. In alternative embodiments, the invention provides methods for treating, ameliorating, slowing the progress of, reversing, or preventing a retroviral infection, an HIV infection, or an HIV-1 infection, or AIDS. In alternative embodiments, the invention provides compositions, including pharmaceutical compositions and formulations, comprising peptides or polypeptide having a sequence as set forth in SEQ ID NO:1 and/or SEQ ID NO:2, and peptidomimetics and bioisosteres thereof.

We performed a retroviral random peptide library-based screen in search for novel inhibitors of HIV-1, and discovered novel peptides, referred to as: Peptide 2, or Thr Arg Arg Val Ala His Asn Ser Glu (TRRVAHNSE) (SEQ ID NO:1); and, Peptide 6, or Trp Arg Gly Ala Ala Met Val Arg Gly (WRGAAMVRG) (SEQ ID NO:2); thus, the invention provides peptides and proteins and formulations comprising them that are and can be used as HIV-1 antivirals agents.

In alternative embodiments, the peptides or polypeptides of the invention are isolated from natural sources, are synthetic, or are recombinantly generated polypeptides. Peptides and proteins of the invention can be recombinantly expressed in vitro or in vivo. The peptides and polypeptides of the invention can be made (synthesized) or isolated using any method known in the art. Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

Peptides or polypeptides of the invention can be glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

Peptides or polypeptides of the invention include use of all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered (e.g., it is ITK-inhibitory).

Peptides or polypeptides of the invention can be comprising any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

Peptides or polypeptides of the invention can be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Peptides or polypeptides of the invention can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, in one aspect under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

Peptides or polypeptides of the invention can be altered by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

Peptides or polypeptides of the invention can be made by solid-phase chemical peptide synthesis methods. For example, assembly of a polypeptides or peptides of the invention can be carried out on a solid support using an Applied Biosystems, Inc. Model 431A™ automated peptide synthesizer. Such equipment provides ready access to the polypeptides or peptides of the invention, either by direct synthesis or by synthesis of a series of fragments that can be coupled using other known techniques.

Peptides or polypeptides of the invention can comprise, in addition to the twenty naturally occurring amino acids, several other classes of alpha amino acids are also known. Examples of these other classes include D-amino acids, N_{α}alkyl amino acids, alpha-alkyl amino acids, cyclic amino acids, chimeric amino acids, and miscellaneous amino acids. These non-natural amino acids have been widely used to modify bioactive polypeptides to enhance resistance to proteolytic degradation and/or to impart conformational constraints to improve biological activity (Hruby et al., Biochem. J. (1990) 268:249-262; Hruby and Bonner, Methods in Molecular Biology (1994) 35:201-240). The most common N_{α}-alkyl amino acids are the N_{α}-methyl amino acids, such as, N_{α}-methyl glycine (i.e., NMeGly, sarcosine, Sar), N_{α}-methyl alanine (i.e., NMeAla), and N_{α}-methyl lysine (i.e., NMeLys). Also included herein are other N_{α}-methyl amino acids including N_{α}-methyl valine (i.e., NMeVal), N_{α}-methyl leucine (i.e., NMeLeu), and N_{α}-methyl phenylalanine (i.e., NMePhe). Examples of alpha-alkyl amino acids include alpha-aminoisobutyric acid (i.e., Aib), diethylglycine (i.e., Deg), diphenylglycine (i.e., Dpg), alpha-methyl proline (i.e., (αMe)Pro), and alpha-methyl valine (i.e., (αMe)Val) (Balaram, Pure & Appl. Chem. (1992) 64:1061-1066; Toniolo et al., Biopolymers (1993) 33:1061-1072; Hinds et al., J. Med. Chem. (1991) 34:1777-1789). Examples of cyclic amino acids include 1-amino-1-cyclopropane carboxylic acid, 1-amino-1-cyclopentane carboxylic acid (i.e., cyclic leucine), aminoindane carboxylic acid (i.e., Ind), tetrahydroisoquinolinecarboxylic acid (i.e., Tic) and tetrahydrocarbolinecarboxylic acid (i.e., Tca) (Toniolo, C., Int. J. Peptide Protein Res. (1990) 35:287-300; Burgess, K., Ho, K. K., and Pal, B. J. Am. Chem. Soc. (1995) 117:3808-3819). Also included are alkenyl and alkynyl containing amino acids such as propargylglycine, dehydroalanine, and the like. Examples of chimeric amino acids include penicillamine (i.e., Pen), combination of cysteine with valine, and 4-mercaptoproline (i.e., Mpt), combination of proline and homocysteine. Example of miscellaneous alpha-amino acids include ornithine (i.e., Orn), 2-naphthylalanine (i.e., 2-Nal), phenylglycine (i.e., Phg), t-butylglycine (i.e., tBug), alpha-ethylglycine (i.e., (αEt)Gly), alpha-n-propylglycine (i.e., (αPr)Gly), alpha-n-butylglycine (i.e., nBug), O-benzylserine (i.e., (OBzl)Ser), p-bromophenylalanine (i.e., pBrPhe), cyclohexylalanine (i.e., Cha), and alpha-amino-2-thiophenepropionic acid (i.e., Thi). In addition to alpha-amino acids, others such as beta amino acids, can also be used in the present invention. Examples of these other amino acids include 2-aminobenzoic acid (i.e., Abz), beta-aminopropanoic acid (i.e., beta-Apr), gamma-aminobutyric acid (i.e., gamma-Abu), and 6-aminohexanoic acid (i.e., epsilon-Ahx).

Generating and Manipulating Nucleic Acids

In alternative embodiments, the invention provides nucleic acids encoding peptides and polypeptides of the invention, comprising e.g., SEQ ID NO:1 and SEQ ID NO:2. The invention can be practiced in conjunction with any method or protocol or device known in the art, which are well described in the scientific and patent literature.

The invention provides “nucleic acids” or “nucleic acid sequences” encoding peptides and polypeptides of the invention, including oligonucleotides, nucleotides, polynucleotides, or any fragments of these, including DNA or RNA (e.g., mRNA, rRNA, tRNA) of genomic or synthetic origin, which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., iRNPs). Nucleic acids encoding peptides and polypeptides can comprise analogues of natural nucleotides, e.g., can be naturally occurring nucleic acids, synthetic nucleic acids or recombinant nucleic acids, or nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. The invention provides for use of deoxyribonucleotide (DNA) or ribonucleotide (RNA) in either single- or double-stranded form. The invention provides for use of nucleic acids containing known analogues of natural nucleotides. The invention provides for use of nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene (methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). The invention provides for use of PNAs containing non-ionic backbones, such as N-(2-aminoethyl) glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). The invention provides for use of nucleic acids including genes, polynucleotides, DNA, RNA, cDNA, mRNA, oligonucleotide primers, probes and amplification products.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

The nucleic acids used to practice this invention, whether RNA, iRNA, siRNA, antisense nucleic acid, cDNA, genomic DNA, vectors, viruses or hybrids thereof, may be isolated from a variety of sources, genetically engineered, amplified, and/or expressed/generated recombinantly. Recombinant peptides or polypeptides generated from these nucleic acids can be individually isolated or cloned and tested for desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Alternatively, nucleic acids can be obtained from commercial sources.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., Molecular Cloning: A Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization with Nucleic Acid Probes, Part I. Theory and Nucleic Acid Preparation, Tijssen, ed. Elsevier, N.Y. (1993).

In alternative embodiments, nucleic acids used to practice the invention, e.g., express peptides or polypeptides of the invention, include, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In practicing the invention, nucleic acids of the invention or modified nucleic acids of the invention, can be reproduced by amplification. Amplification can also be used to clone or modify the nucleic acids used to practice the invention. Thus, the invention provides amplification primer sequence pairs for amplifying nucleic acids of the invention. One of skill in the art can design amplification primer sequence pairs for any part of or the full length of these sequences.

Bioisosteres of Compounds of the Invention

In alternative embodiments, the invention also provides bioisosteres of peptides of the invention, e.g., polypeptides having a sequence as set forth in SEQ ID NO:1 or SEQ ID NO:2. In alternative embodiments, bioisosteres of the invention are compounds of the invention comprising one or more substituent and/or group replacements with a substituent and/or group having substantially similar physical or chemical properties which produce substantially similar biological properties to a compound of the invention, or stereoisomer, racemer or isomer thereof. In one embodiment, the purpose of exchanging one bioisostere for another is to enhance the desired biological or physical properties of a compound without making significant changes in chemical structures.

For example, in one embodiment, bioisosteres of compounds of the invention are made by replacing one or more hydrogen atom(s) with one or more deuterium or fluorine atom(s), e.g., at a site of metabolic oxidation; this may prevent metabolism (catabolism) from taking place. Because the deuterium or fluorine atom is similar in size to the hydrogen atom the overall topology of the molecule is not significantly affected, leaving the desired biological activity unaffected. However, with a blocked pathway for metabolism, the molecule may have a longer half-life or be less toxic, and the like.

### Formulations and Pharmaceutical Compositions

In alternative embodiments a composition of the invention, e.g., a peptide or polypeptide comprising SEQ ID NO:1 and/or SEQ ID NO:2, is formulated for administration by any or a variety of means including orally, parenterally, by inhalation spray, nasally, topically, intrathecally, intrathecally, intracerebrally, epidurally, intracranially or rectally. Formulations of the invention can comprise pharmaceutically acceptable carriers, adjuvants and vehicles. In alternative embodiments, composition of this invention, or a composition used to practice the methods of this invention, are formulated for parenteral administration, including administration intrathecally, intracerebrally or epidurally (into a intrathecal, intracerebral, epidural space), subcutaneously, intravenously, intramuscularly and/or intraarterially; e.g., by injection routes but also including a variety of infusion techniques. Intraarterial, intrathecal, intracranial, epidural, intravenous and other injections as used in some embodiments can include administration through catheters or pumps, e.g., an intrathecal pump, or an implantable medical device (which can be an intrathecal pump or catheter).

In alternative embodiments a compound of the invention, or a composition used to practice the methods of this invention, can be formulated in accordance with a routine procedure(s) adapted for a desired administration route. Accordingly, in alternative embodiments compounds used to practice the invention are formulated or manufactured as lyophilates, powders, lozenges, liposomes, suspensions, solutions or emulsions in oily or aqueous vehicles, and can contain formulatory agents such as suspending, stabilizing and/or dispersing agents.

In alternative embodiments a compound of the invention, or a composition used to practice the methods of this invention, can be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient (e.g., a composition of the invention) can be in powder form for constitution with a suitable vehicle, e.g., sterile pyrogen-free water, before use. Suitable alternative and exemplary formulations for each of these methods of administration can be found, e.g., in Remington: The Science and Practice of Pharmacy, A. Gennaro, ed., 20th edition, Lippincott, Williams & Wilkins, Philadelphia, Pa.

In alternative embodiments a compound of the invention, or a composition used to practice the methods of this invention, can be formulations for parenteral administration comprising any common excipient, e.g., sterile water or saline, a polyalkylene glycol such as a polyethylene glycol, an oil of synthetic or vegetable origin, a hydrogenated naphthalene and the like. In alternative embodiments, a compound used to practice the invention can be a biocompatible, biodegradable lactide polymer, a lactide/glycolide copolymer, or polyoxyethylene-polyoxypropylene copolymers can be useful excipients to control the release of active compounds.

In alternative embodiments, a composition of the invention, or a composition used to practice the methods of this invention, is administered using parenteral delivery systems such as ethylene-vinyl acetate copolymer particles, osmotic pumps, implantable infusion systems, intrathecal catheters, pumps and implants, and/or use of liposomes. Formulations for parenteral administration can also include glycocholate for buccal administration, methoxysalicylate for rectal administration, or citric acid for vaginal administration. Formulations for inhalation administration can contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally.

In alternative embodiments, formulations of the invention, or a composition used to practice the methods of this invention, are administered intranasally. When given by this route, examples of appropriate dosage forms are a nasal spray or dry powder, as is known to those skilled in the art. For example, a nasal formulation can comprise a conventional surfactant, generally a non-ionic surfactant. When a surfactant is employed in a nasal formulation, the amount present will vary depending on the particular surfactant chosen, the particular mode of administration (e.g. drop or spray) and the effect desired.

In alternative embodiments, a pharmaceutical composition of the invention, or a composition used to practice the methods of this invention, is in the form of a sterile injectable preparation, such as a sterile injectable aqueous or oleaginous suspension. This suspension may be formulated according to the known art using those suitable dispersing or wetting agents and suspending agents. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In alternative embodiments, sterile fixed oils are conventionally be employed as a solvent or suspending medium. For this purpose any bland fixed oil may be employed including synthetic mono- or diglycerides. In alternative embodiments, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule (ampoule) or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

In alternative embodiments, formulations of the invention, or a composition used to practice the methods of this invention, further comprise aqueous and non-aqueous sterile injection solutions that can contain (comprise) antioxidants, buffers, bacteriostats, bactericidal antibiotics and solutes that render the formulation isotonic with the bodily fluids of the intended recipient; and/or aqueous and non-aqueous sterile suspensions, which can include suspending agents and thickening agents.

In alternative embodiments, compounds of the invention, or a composition used to practice the methods of this invention, are formulated for topical administration, e.g., in the form of a liquid, lotion, cream or gel. Topical administration can be accomplished by application directly on the treatment area. For example, such application can be accomplished by rubbing the formulation (such as a lotion or gel) onto the skin of the treatment area, or by a spray application of a liquid formulation onto the application or treatment area.

In alternative embodiments, formulations of the invention, or a composition used to practice the methods of this invention, comprise a bioimplant or a bioimplant material, and also can be coated with a compound of the invention or another compounds so as to improve interaction between cells and the implant.

In alternative embodiments, formulations of the invention, or a composition used to practice the methods of this invention, comprise minor amounts of wetting or emulsifying agents, or pH buffering agents.

In alternative embodiments, formulations of the invention, or a composition used to practice the methods of this invention, are formulated as a suppository, with traditional binders and carriers such as triglycerides.

In alternative embodiments, formulations of the invention, or a composition used to practice the methods of this invention, comprise oral formulations such as tablets, pills, troches, lozenges (see, e.g., as described in U.S. Pat. No. 5,780,055), aqueous or oil suspensions, dispersible powders or granules, emulsions, hard or soft capsules or geltabs, gels, jellies, syrups and/or elixirs. Compositions intended for oral use may be prepared according to any method known to the art for the manufacture of pharmaceutical compositions and such compositions may contain one or more agents including sweetening agents, taste-masking agents, flavoring agents, coloring agents and preserving agents, in order to provide a palatable preparation. Oral formulations can include standard carriers such as pharmaceutical grades of mannitol, lactose, starch, magnesium stearate, polyvinyl pyrrolidone, sodium saccharine, cellulose, magnesium carbonate, etc. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In alternative embodiments, formulations for oral use are hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

In alternative embodiments, formulations of the invention, or a composition used to practice the methods of this invention, comprise aqueous suspensions comprising the active material (e.g., a compound of this invention) in admixture with excipients suitable for the manufacture of aqueous suspensions. Exemplary excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

In alternative embodiments, formulations of the invention, or a composition used to practice the methods of this invention, comprise oil suspensions that can be formulated by suspending the active ingredient (e.g., a compound of this invention) in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

In alternative embodiments, pharmaceutical formulations comprising the compounds of the invention, or a composition used to practice the methods of this invention, include an agent which controls release of the compound, thereby providing a timed or sustained release compound.

Carriers

In alternative embodiments, pharmaceutically acceptable carriers for manufacturing or formulating compounds of this invention (e.g., a peptide or polypeptide comprising SEQ ID NO:1 and/or SEQ ID NO:2), or a composition used to practice the methods of this invention, comprise aqueous or non-aqueous solutions, suspensions, emulsions and solids. Examples of non-aqueous solvents suitable for use as disclosed herein include, but are not limited to, propylene glycol, polyethylene glycol, vegetable oils such as olive oil, and injectable organic esters such as ethyl oleate. In alternative embodiments, aqueous carriers can comprise water, ethanol, alcoholic/aqueous solutions, glycerol, emulsions and/or suspensions, including saline and buffered media. Oral carriers can be elixirs, syrups, capsules, tablets and the like.

In alternative embodiments, liquid carriers are used to manufacture or formulate compounds of this invention, or a composition used to practice the methods of this invention, including carriers for preparing solutions, suspensions, emulsions, syrups, elixirs and pressurized compounds. The active ingredient (e.g., a peptide or polypeptide comprising SEQ ID NO:1 and/or SEQ ID NO:2) can be dissolved or suspended in a pharmaceutically acceptable liquid carrier such as water, an organic solvent, a mixture of both or pharmaceutically acceptable oils or fats. The liquid carrier can comprise other suitable pharmaceutical additives such as solubilizers, emulsifiers, buffers, preservatives, sweeteners, flavoring agents, suspending agents, thickening agents, colors, viscosity regulators, stabilizers or osmo-regulators.

In alternative embodiments, liquid carriers used to manufacture or formulate compounds of this invention comprise water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellant.

In alternative embodiments, solid carriers are used to manufacture or formulate compounds of this invention, or a composition used to practice the methods of this invention, including solid carriers comprising substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

In alternative embodiments, parenteral carriers are used to manufacture or formulate compounds of this invention, or a composition used to practice the methods of this invention, including parenteral carriers suitable for use as disclosed herein include, but are not limited to, sodium chloride solution, Ringer's dextrose, dextrose and sodium chloride, lactated Ringer's and fixed oils. Intravenous carriers can comprise fluid and nutrient replenishers, electrolyte replenishers such as those based on Ringer's dextrose and the like. Preservatives and other additives can also comprise, for example, antimicrobials, antioxidants, chelating agents, inert gases and the like.

In alternative embodiments, carriers used to manufacture or formulate compounds of this invention, or a composition used to practice the methods of this invention, can be mixed as needed with disintegrants, diluents, granulating agents, lubricants, binders and the like using conventional techniques known in the art. The carriers can also be sterilized using methods that do not deleteriously react with the compounds, as is generally known in the art.

The invention also provides articles of manufacture and kits containing (comprising) compounds of this invention, or a composition used to practice the methods of this invention, including pharmaceutical compositions and formulations. By way of example only a kit or article of manufacture can include a container (such as a bottle) with a desired amount of a compound (or pharmaceutical composition of a compound) described herein. Such a kit or article of manufacture can further include instructions for using the compound (or pharmaceutical composition of a compound) described herein. The instructions can be attached to the container, or can be included in a package (such as a box or a plastic or foil bag) holding the container.

The compounds of the invention, or a composition used to practice the methods of this invention, can be delivered to the body or targeted to a specific tissue or organ (e.g., a muscle or a brain) by any method or protocol, e.g., including ex vivo “loading of cells” with a composition of the invention; where the “loaded cell” is the administered intramuscularly, or intrathecally, intracerebrally, or epidurally into the central nervous system (CNS), e.g., as described in U.S. Pat. App. Pub. No. 20050048002.

In alternative embodiments, compounds of the invention, or a composition used to practice the methods of this invention, are first lyophilized and then suspended in a hydrophobic medium, e.g., comprising aliphatic, cyclic or aromatic molecules, e.g., as described in U.S. Pat. App. Pub. No. 20080159984.

### Development of a High-Throughput Assay for HIV-1 Envelope Maturation

In alternative embodiments, the invention provides high-throughput cell-based assays for the screening of compounds or drugs against HIV-1 gp160 envelope protein processing. In alternative embodiments, assays of the invention comprise use furin (also known as PACE (Paired basic Amino acid Cleaving Enzyme; an enzyme which belongs to the subtilisin-like proprotein convertase family; members of this family are proprotein convertases that process latent precursor proteins into their biologically active products), which recognizes the gp160 envelope protein, as a novel target for drug development against HIV-1 (the viral gp160 envelope protein is the only one of the viral proteins not processed by PR; instead, furin and similar host peptidases are responsible for processing gp160 into gp120 and gp41 in the Trans-Golgi Network (TGN) luminal compartment, and processing of gp160 is absolutely necessary for the production of infectious viral particles).

We engineered a scaffold molecule targeted to the Endoplasmic Reticulum (ER)/TGN for classical transport pathway to the cell surface. For that purpose, we exploited a murine CD8a (LYT2) FLAG-tagged molecule that we have previously engineered and is easily recognized on the cell surface by flow cytometry. This molecule was further engineered to contain two tags: FLAG and HA, flanking the gp120/41 recognition/cleavage site. The double-tagged construct was then fused upstream of the LYT2 scaffold for proper localization of the gp120/41 boundary in the ER/TGN lumen. The engineered scaffold allows the discrimination of cleaved and non-cleaved events based on the cell surface expression of one tag, HA, or two, FLAG and HA, respectively.

FLAG expression on the cell-surface can thus be used as a biosensor for the activity of furin and similar host peptidases in a flow cytometry-based manner. In alternative embodiments, the high throughput assays of the invention are used to screen for compounds or drugs targeting HIV-1 envelope processing, as well as elucidate the still unclear mechanisms of gp160 maturation. In alternative embodiments, the high throughput assays of the invention use HIV-1 envelope maturation as a novel target for the inhibition of HIV infection.

In alternative embodiments, the invention provides a cell-based assay for the identification of protease recognition/cleavage sites within the host proteome, and methods for screening for their discovery. In one embodiment, HIV-1 PR is used, and this specific screen is performed in a T-cell line to mimic the natural milieu of infection. In alternative embodiments, many cellular targets can be identified using the cell-based assay of the invention performed in an appropriate milieu. In alternative embodiments assays of the invention, coupled with the screen, can be used as a platform for the search of cellular substrates for viral proteases, no matter the virus, provided the viruses rely, at least in part, on their own proteases. Revealing the host targets for viral proteases will in turn elucidate biological pathways involved in the establishment of infection and the understanding of virus/host interactions.

Accordingly, the invention provides methods and compositions, including chimeric recombinant proteins, nucleic acids that encode them, and cells and kits comprising them, to screen for compositions, e.g., small molecule drugs, that can modulate, e.g., inhibit or enhance, viral proteases, including retroviral (e.g., HIV) proteases such as furin.

In one embodiment, the invention engineers a protein scaffold bearing the protease cleavage site on the cell surface of a mammalian cell (e.g., a lymphocyte such as a T cell, or a hepatocyte). In one embodiment, the invention expresses, or co-expresses, a protease, e.g., a furin, and a scaffold used as a target, in an inducible manner (the protease, the scaffold, or both can be expressed via an inducible mechanism, e.g., an inducible transcriptional regulator).

In one embodiment, the invention provides assays that can be adapted for a high throughput manner using e.g. flow cytometry such as FACS, and can discriminate between active and non-active or blocked protease. In one embodiment, the invention provides assays that can be easily adapted for high throughput screening. In one embodiment, the invention provides assays of this invention can be used to screen for novel protease inhibitors.

In one embodiment, the invention provides assays of this invention adapted for the screen of random peptide libraries or chemical compounds for drug discovery.

The invention will be further described with reference to the following examples; however, it is to be understood that the invention is not limited to such examples.

### EXAMPLES

### Example 1

### Peptide Inhibitors Against HIV-1

The invention provides peptide inhibitors of HIV-1, and compositions, formulations, and pharmaceutical compositions comprising same, and methods of making and using them.

We performed a retroviral random peptide library-based screen in search for novel inhibitors of HIV-1. The library was targeted to the nucleus in order to increase the chances of finding novel inhibitors against nuclear processes involved in the viral life cycle. These include mainly nuclear transport of the pre-integration complex and integration of the viral genome within the host genome by the viral enzyme Integrase. We have found two peptides, referred to as Peptide 2, or Thr Arg Arg Val Ala His Asn Ser Glu (TRRVAHNSE) (SEQ ID NO:1) and Peptide 6, or Trp Arg Gly Ala Ala Met Val Arg Gly (WRGAAMVRG) (SEQ ID NO:2); thus, the invention provides peptides and proteins and formulations comprising them that are and can be used as HIV-1 antivirals agents. Based on their sequence homology to known proteins (see Table 1), they contain high similarity with other proteins involved in DNA binding, recognition or cleavage.

Screening Process:

A retroviral random peptide library localized to the nucleus, 3XF(GGS)₃ NLS RPL, was used to search for novel inhibitors of HIV-1. In order to screen a population of SupT1 expressing this library, a self-inactivating pseudo-typed HIV-1 carrying a pCMV-GFP expression cassette was utilized. The 3XF(GGS)₃ NLS RPL SupT1 cells and cells expressing pBMN.i.mCherry were then screened by infection with sinHIV-GFP. 72 hours post-infection, the cells were sorted by Fluorescence Activated Cell Sorting (FACS) based on the expression of GFP, retaining and expanding GFP-negative cells in culture. Once expanded, the non-fluorescent cells were re-infected with sinHIV-GFP, and the process repeated until a decrease in the infection rate of library expressing cells was significantly lower than the control cells. Following 12 rounds of infection, selection of non-fluorescent cells and amplification of the 3XF(GGS)₃ NLS RPL expressing SupT1 cells, eight clones were isolated, and expanded in culture. Of the eight, clones 2 and 6 exhibited the largest decrease in sinHIV-GFP infection rate, as compared to the mCherry control.

Potential Candidates:

Peptide 6: Trp Arg Gly Ala Ala Met Val Arg Gly (WRGAAMVRG) shares a high degree of homology with the RuvC Holliday junction endonuclease of Acidothermus cellulolyticus 11B. HIV-1 IN contains a αβ-fold containing a central five-stranded mixed fl-sheet surrounded by α-helices on both sides in the same topological order as Escherichia Coli RuvC, E. Coli RNase H and the HIV-1 RNase H domain of Reverse Transcriptase. All four structures exhibit the folding topology of many nucleotide-binding proteins across many organisms.

Peptide 2: Thr Arg Arg Val Ala His Asn Ser Glu (TRRVAHNSE) was also predicted to be homologous to an endonuclease/exonuclease/phosphatase family domain 1 protein of Ciona intestinalis.

TABLE 1
Sequences with significant alignments
as determined by Protein BLAST ®

| | | Protein | Max | E |
|---|---|---|---|---|
| Peptide | Protein Name | Sequence | Score | value |
| Clone 2 | Putative tail fiber protein | RVVAHNSD | 25.7 | 163 |
| TRVVAHNSE | [Brochothrix phage BL3] | (SEQ ID NO: 3) | | |
| | Predicted: similar to | TRVVAHN | 25.2 | 218 |
| | endonuclease/exonuclease/ | (SEQ ID NO: 4) | | |
| | phosphatase family domain | | | |
| | containing 1 [Ciona intestinalis] | | | |
| | Type VI secretion protein, family | TKVVAHNSD | 25.2 | 219 |
| | [Campylobacter rectus RM3267] | (SEQ ID NO: 5) | | |
| | hypothetical protein PSF113_0324c | RVVAQNSE | 24.8 | 289 |
| | [Pseudomonas fluorescens F113] | (SEQ ID NO: 6) | | |
| | Arginase [Rhodobacterales | TRVIAHTSE | 24.8 | 290 |
| | bacterium HTCC2083] | (SEQ ID NO: 7) | | |
| Clone 6 | Holliday junction endonuclease | WRGAAMAR | 26.9 | 0.007 |
| WRGAAMVRG | RuvC [Acidothermus cellulolyticus | (SEQ ID NO: 8) | | |
| | 11B] | | | |
| | Hypothetical protein JDM601_2017 | WKDRGAAMVR | 26.1 | 0.012 |
| | [Mycobacterium sp. JDM601] | (SEQ ID NO: 9) | | |
| | ABC transporter-like protein | WRGAALCMVR | 26.1 | 0.013 |
| | [Verminephrobacter aporrectodeae | (SEQ ID NO: 10) | | |
| | subsp. tuberculatae At4] | | | |
| | DNA | WRGAVCAMVR | 26.1 | 0.013 |
| | helicase/exodeoxyribonuclease V | (SEQ ID NO: 11) | | |
| | subunit beta [Geobacter | | | |
| | metallireducens GS-15] | | | |
| | Cation efflux protein | WRGRAAMVKG | 25.7 | 0.017 |
| | [Marinobacter aquaeolei VT8] | (SEQ ID NO: 12) | | |

### Example 2

### High-Throughput Assay for HIV-1 Envelope Maturation

In alternative embodiments, the invention provides cell-based assays for the identification of protease recognition/cleavage sites within a host proteome.

While viral protease recognition/cleavage sites have been identified within the host proteome, few if any assays have been developed to identify those targets in a cellular milieu. To address this issue, we developed a cell-based assay for the identification of protease recognition/cleavage sites within the host proteome, and perform a screen for their discovery. HIV-1 protease (PR) will be used as proof of principle, and this specific, exemplary screen will be performed in a T-cell line to mimic the natural milieu of infection. Assays of the invention can be used to identify cellular targets, and the robust cell-based assay of the invention, performed in an appropriate milieu, will enhance and facilitate their discovery. While we used for HIV-1 PR as a proof of principle, assays of the invention coupled with the screen can be used as a platform for the search of cellular substrates for viral proteases, no matter the virus, provided the viruses rely, at least in part, on their own proteases. Revealing the host targets for viral proteases will in turn elucidate biological pathways involved in the establishment of infection and the understanding of virus/host interactions.

HIV-1 PR, or proteases in general, have functionalities other than the expected enzymatic cleavage, and many of their binding partners might not serve as substrates. A protein-protein interaction based approach such as the one described by Jager et al, 2012 for HIV-1 PR can reveal binding partners, but may not reveal all the cleavable substrates of PR. While the interactome approach will most likely uncover high affinity binders, in cases where PR cleaves a substrate with very high efficiency the substrate would be readily destroyed, and thus not detected through protein-protein interaction. The assays of this invention can reveal such brief interactors; whether the substrate is cleaved in an efficient manner or not, it will be discriminated from un-cleaved events.

Proteomics approaches for the interactome of virus/host proteins have also been performed for other retroviruses including Human T-Lymphotropic virus or viruses from families such as the Herpesviridae Epstein-Barr virus, the Togaviridae sindbis virus or the Flaviviridae Dengue virus. Practicing assays of this invention can find cellular substrates previously described by Jager et al, 2012, and others, and can also detect and reveal novel cellular substrates. Each novel target can unravel important biological functions leading to the establishment of HIV-1 infection. As exemplary assays and screens of the invention can be performed in or using various cellular milieu, screens of the invention can reveal previously unrecognized but biologically significant targets.

For example, for the search of HIV-1 PR cellular targets, an exemplary assay of the invention can be performed in T-cells, whereas for the search of HCV NS3/4A cellular targets, assays of the invention should be performed in hepatocytes. Importantly, although one exemplary assay of the invention utilizes HIV-1 PR as proof of principle, the platform of the invention will be suitable for proteases of any virus of interest provided that they rely, at least partially, on the processing of their proteome by their own proteases. Moreover, assays of the invention are a tool for the study of protease activity, and will allow search for viral and/or host factors that enhance or facilitate its activity in a simple and straightforward manner. Assays of the invention are an asset for the Pharmaceutical-Biotechnology industry as they can be used to corroborate the inhibitory effect of approved or candidate drugs against proteases based on their ability to inhibit the cleavage of the host factors as well. Robustness of assays of the invention, their quantitative fluorescence-based nature coupled with flow cytometry analysis, all make assays of the invention a perfect tool for proteomics high throughput discovery of cellular cleavable substrates by viral proteases.

In one embodiment, assays of the invention monitor HIV-1 protease (PR) to cellular substrates of viral proteases. In one embodiment, assays of the invention search for novel host substrates. In one embodiment, a SupT1 T-cell line cDNA library is introduced in the context of the assay. Hits obtained are corroborated for their potential as true cleavable substrates.

In one embodiment, assays of the invention identify substrates of PR; assays use one or more putative substrates of HIV-1 protease (PR), rather than PR itself. Known PR substrates can be used as controls to corroborate the robustness and utility of the assay for the search of novel candidate substrates. Two exemplary embodiments are: in one embodiment (scenario) #1, PR is supplied in trans in an independent inducible vector while the Gal4-substrate fusion will be supplied constitutively; in another embodiment (scenario) #2, PR is supplied in cis as part of the Gal4-substrate fusion.

In one embodiment: Construction of cDNA fragment libraries: Two libraries are be constructed from the cDNA of SupT1 T-cells. They can differ in the average size of their segments: approximately 500 base pairs (bp) and an approximately 2000 bp-long library. In alternative embodiments, the libraries are engineered as part of a Gal4 retroviral vector, and can be stably expressed in mammalian cells.

In one embodiment: Screen for putative novel PR substrates: Cells are utilized for the screening of PR substrates. Two different, alternative embodiments: (1) (scenario #1) the first is a negative screen and is based on the expression of PR in trans, in an independent inducible construct; (2) (scenario #2) the second embodiment is a positive screen and is based on the inducible expression of PR in cis, as part the Gal4 fusion. Whether scenario #1 or scenario #2 is chosen for any particular assay can depend on: if PR works well in trans and allows clear discrimination between cleaved and non-cleaved known targets, scenario #1 is chosen; or, if in another particular assay better discrimination is obtained with PR in cis as part of the Gal4 fusion, scenario #2 is chosen. The two exemplary assays of the invention, the two screens, are not incompatible, and can be performed concurrently.

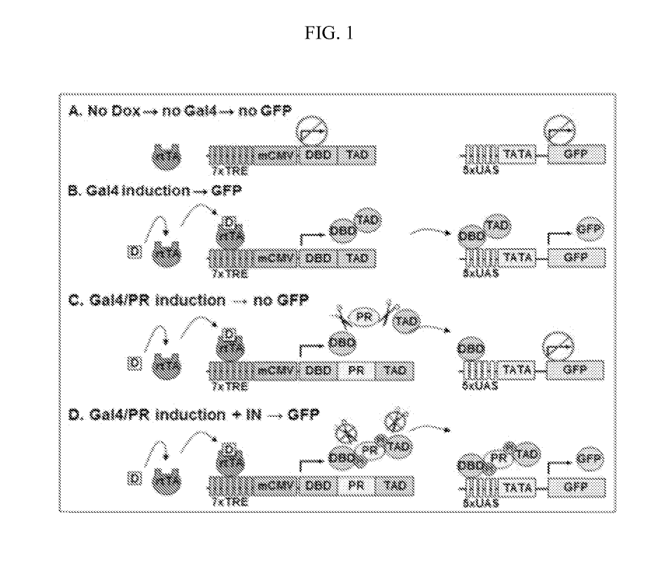

Alternative embodiments adapt the exemplary assay as illustrated in FIG. 1, which was developed for the monitoring of PR activity, to alternative exemplary assays that search for PR substrates instead. Most of the preliminary data is based on the assay developed for HIV-1 PR because it demonstrates beyond all doubt the feasibility of exemplary the PR substrate assay and its utility for screening.

FIG. 1 describes an exemplary assay successfully implemented with wild-type HXB2 consensus PR. In the assay, the Gal4/PR fusion is expressed in an inducible manner via a Tetracycline-On system (Tet-On), in order to avoid PR expression when not required, minimizing potential cytotoxic side effects prior to the screen. For this purpose, the reverse tetracycline transactivator (rtTA) enables induction of Gal4/PR expression only upon the addition of Tet or doxycycline (Dox). GFP, which serves as sensor for PR activity, is expressed only when PR/Gal4 expression is induced in the presence of a PR inhibitor (PI).

FIG. 1: overview of the assay: FIG. 1A. Wild type Gal4 as control, no Dox. Without Dox, rtTA cannot bind to the Tet-responsive element (TRE) and induce Gal4 expression. Consequently, no GFP expression is expected from the reporter construct. FIG. 1B: in the presence of Dox (the “D” in the grey box), Gal4 expression is induced, activating GFP expression upon binding of rtTA to the 5×UAS element of the reporter construct. FIG. 1C: the Gal4/PR fusion-based system. In the presence of Dox, Gal4/PR is expressed; however, its catalytic activity results in the separation of the Gal4 domains, and thus no GFP expression. FIG. 1D: same scenario as in C but in the presence of PI (orange circles). Gal4/PR fusion remains intact, resulting in the induction of GFP expression. (Dox: doxycycline, PI: protease inhibitor)

Design of Lentiviral Constructs for the Inducible Expression of PR in T-Cells:

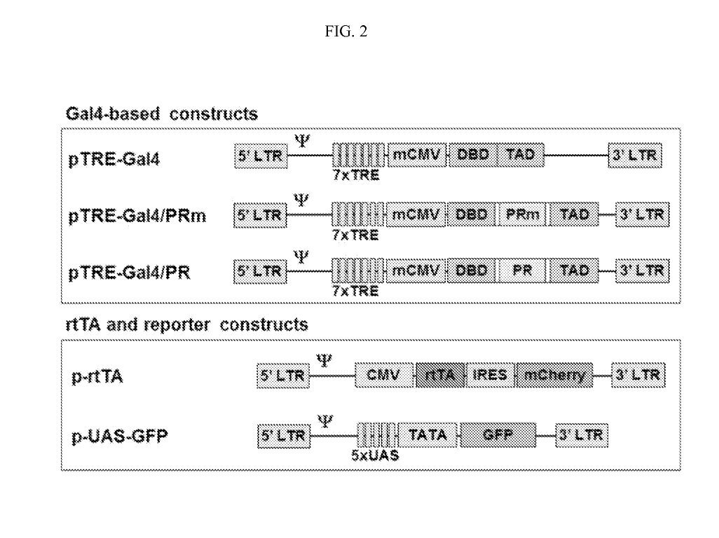

In order to transfer the assay elements into SupT1 T-cells, and express them in a stable manner, we utilized retroviral technology. The exemplary system outlined in FIG. 1 relies on three kinds of vectors, as depicted in FIG. 2: a retroviral rtTA expression vector (p-rtTA), a lentiviral reporter vector with the UAS Gal4 responsive element (five copies) upstream of a minimal Cytomegalovirus promoter driving GFP expression (pUAS-GFP) and the inducible Gal4-based lentiviral vectors; control (pTRE-Gal4), and mutant (D25A) and wild-type PR fusions (pTRE-Gal4/PRm and pTRE-Gal4/PR).

The UAS reporter cassette was inserted into an HIV-based self-inactivating vector, with most of the 3′ HIV Long Terminal Repeat (LTR) U3 sequence deleted to ensure that the reporter activity is based only on the 5×UAS element and not the viral promoter. In addition, this ensures no background reporter activity in the absence of inhibitor. The same vector was utilized to insert the Gal4-based constructs. These vectors, being of retroviral/lentiviral nature, enable stable transfer of the constructs into mammalian cells for further selection of the clones of interest. In addition, their inducible nature allow for inducible PR expression, thus circumventing the possible cytotoxicity of PR in mammalian cells.

To obtain an inducible cell line, we utilized the Tet-On system (adapted from Clontech), where rtTA binds to and activates expression from the Tet Response Element (TRE) only in the presence of the inducer (Tet or Dox). For this purpose, we constructed two retroviral/lentiviral vectors. One of the vectors constitutively expresses the rtTA element coupled to mCherry through an internal ribosome entry site (IRES), allowing corroboration of rtTA expression based on red fluorescence (p-rtTA plasmid in FIG. 2) (a Lyt2; mouse CD8, marker substituting mCherry was used in subsequent experiments for the selection of rtTA-expressing cells by flow cytometry). The other vector is used for the inducible-expression of the Gal4-based fusion proteins, which were inserted downstream seven copies of TRE, in order to activate Gal4, Gal4/PRm or Gal4/PR (pTRE vectors in FIG. 2).

FIG. 2: Retroviral constructs utilized in the exemplary assay as described in FIG. 1: constructs are utilized to generate infectious particles for the transduction of SupT1 cells with the various assay elements including the Gal4-based vectors: Gal4, Gal4/PRm and Gal4/PR and rtTA, and GFP reporter constructs. (LTR: Long Terminal Repeat, . . . packaging signal).

Insertion of the HIV-1 PR Sequence Between the Gal4 Domains Disrupts Gal4 Activity Unless Inhibited:

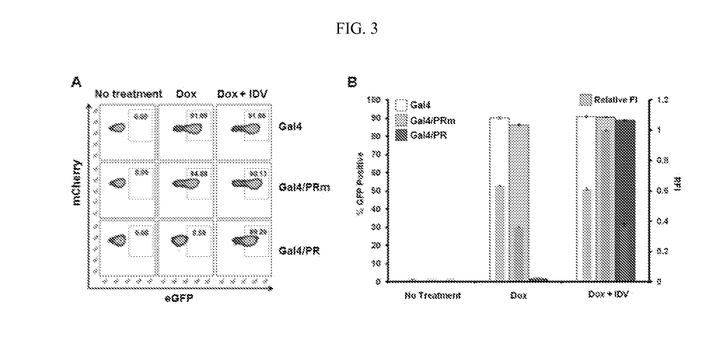

When SupT1 cells were infected with lentiviral particles containing reporter vector or Gal4 alone, no detectable GFP expression was observed (data not shown). Importantly, in cells carrying reporter and rtTA, addition of 1 μg/mL Dox resulted in substantial GFP expression by Gal4, as expected (FIG. 3A, top panels).

Next, we corroborated that insertion of PR between DBD and TAD of Gal4 does not disrupt its transcriptional activity. To test this, we introduced D25A, an inactive version of PR, referred to as PR mutant (PRm). The catalytic core of PR resides at the Asp, Thr, Gly triad of PR, and mutations at Asp25 have previously been shown to result in the loss of its catalytic activity. In addition to the PR sequence, based on the HXB2 consensus genome, 22 upstream and 32 downstream amino acids (to include the PR cleavage sites), were also included. PR D25A should not be able to separate the domains, which would disrupt the ability of DBD and TAD to work in conjunction and thus activate GFP expression. Indeed, when clones expressing reporter and rtTA together with PRm/Gal4 were induced with Dox, GFP expression was detected (FIG. 3, middle panels (FIG. 3A) and columns (FIG. 3B)). Although the induction of GFP expression by the Gal4/PRm fusion was slightly lower than that of Gal4 alone, the level of activation was clear, corroborating that the insertion, per se, of the PR sequence between the Gal4 domains does not have a big impact on Gal4 function. Most importantly, when the experiment was repeated with Gal4/PR, no GFP expression was observed even after the addition of Dox (FIG. 3A, lower panels). However, Indinavir (10 μM) inhibited PR, allowing robust GFP induction (FIG. 3, left panels (FIG. 3A) and columns (FIG. 3B)). Moreover, a screen performed with the eight other FDA approved inhibitors showed similar results, proving that the assay was not specific to Indinavir. Importantly, the assay was shown to be robust and reproducible, with a Z-factor of 0.87 (calculated as described by Zhang et al.).

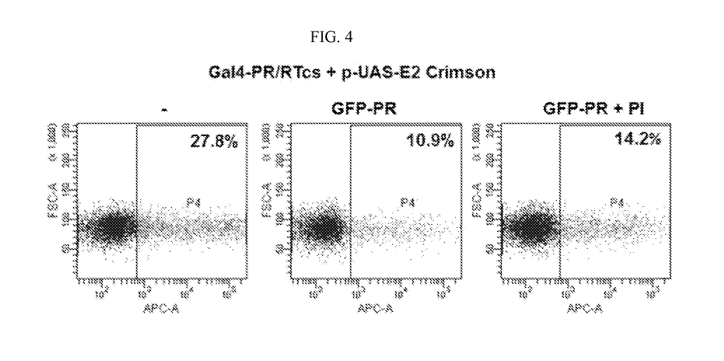

This data clearly demonstrates the robustness and quantitative nature of this exemplary assay for the monitoring of HIV-1 PR activity. Alternative embodiments adapt this exemplary assay to PR substrates: in a transient transfection experiment performed with PR/RT, one of the known HIV-1 recognition/cleavage sites (FIG. 4), the PR/RT boundary was introduced between the Gal4 domains. 293T cells were transfected with p-UAS-GFP reporter, rtTA vector and inducible PR expression vector.

While a transient expression experiment is not expected to show substantive results, it can demonstrate proof of principle. Importantly, the affinity of PR to the PR/RT cleavage is not considered high, further validating the assay for substrate recognition. Partial cleavage (as seen by reduction in green fluorescence, FIG. 4, mid panel) was indeed observed in the presence of PR. Importantly, when darunavir (e.g., PREZISTA™) was added as PI, the trend was reversed (FIG. 4, right panel) showing that the reduction in green fluorescence was indeed due to PR activity. The assay can be modified, e.g., where individual clones carry all the elements of the assay such that the results can be more robust.

FIG. 3: Analysis of selected clones: FIG. 3A. Flow cytometry analysis of SupT1 clones expressing Gal4-(top panels), PRm/Gal (middle panels) or PR/Gal4 (bottom panels) were analyzed 72 hours post-infection following treatment with DMSO, DMSO+1. g/mL Dox, or 1. g/mL Dox+10.M IDV; FIG. 3B. Quantification of % of green fluorescence and relative fluorescence intensity (RFI).

FIG. 4: Preliminary transient experiments in 293T cells with PR supplied in trans: Flow cytometry analysis of cells transfected with Gal4-RT/IN substrate fusion and GFP reporter (left panel, in the presence of PR (mid panel) or PR and PI (right panel).

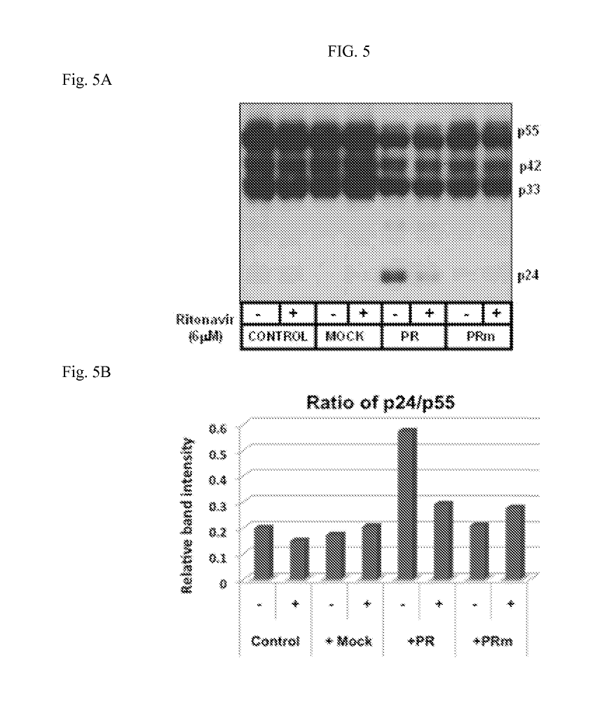

FIG. 5: Biochemical Assay for PR activity. FIG. 5A. HEK 293T cells transfected with p96ZM651gag-opt (lanes 1 and 2) and pcDNA empty vector (lanes 3 and 4) or wild-type PR (lanes 5 and 6) or the inactive PR mutant (lanes 7 and 8) in the presence or absence of PI. Transfected cells were analyzed 48 hours post-transfection with anti-p24 antibody. The gag precursor (p55) and the end product (p24) are shown along with the intermediate products (p42 and p33). FIG. 5B. Quantification of the Western blots by IMAGEJ™ (ImageJ™) software. Band intensities are plotted as a ratio of p24 to p55.

We performed a biochemical assay to assess PR activity based on its ability to cleave one of its natural substrates—the p55 Gag poly-protein. For this purpose, p96ZM651gag-opt (from the NIH AIDS reagent program) with humanized codons for optimal expression, was used as substrate. Cells were co-transfected with p55 and PR in the presence or absence of Ritonavir as PI. Cells were then analyzed by western blot 48 hours post-transfection with antibodies against HIV p24, capsid (NIH AIDS reagent program). Active PR cleaves p55 to produce p24, along with some intermediate products, prominently p44 and p32. However, when PR is inhibited, poly-protein processing is hindered, resulting in the accumulation of the precursor and the intermediates. Similar experiments can be performed with PR in the presence of a tagged putative substrate.

One exemplary embodiment comprises an assay for the monitoring of the activity of HIV-1 PR performed in a T-cell-based context to mimic the milieu of HIV-1 infection. In this exemplary assay, HIV-1 PR is fused between the DNA-binding domain (DBD) and the trans activation domain (TAD) of the Gal4 prototypic transcription factor. PR fused within Gal4 auto-catalytically cleaves itself, leaving behind the two non-functional Gal4 domains. However, when inhibited, the fusion protein remains intact, retaining its function as transcription factor. The intact PR/Gal4 fusion activates expression of the green fluorescence reporter gene (GFP), which serves as a biosensor for PR activity in an inverse manner. The robustness and reproducibility of the assay, which can be quantitatively analyzed, makes it easily adaptable to PR substrates specifically addressing whether substrates are cleaved or not.

Rationale: The majority of assays developed so far have been designed in vitro and monitor the activity of proteases in bacterial or yeast cells. Assays that are designed to find protease substrates rely on a specific known substrate, rather focus on protease specificity, and are thus not intended to search for novel or unknown substrates. In vitro assays do not represent the natural milieu and are biased, as they specifically target the catalytic domain of protease, while mammalian cell-based assays can target any protease surface needed for its activity.

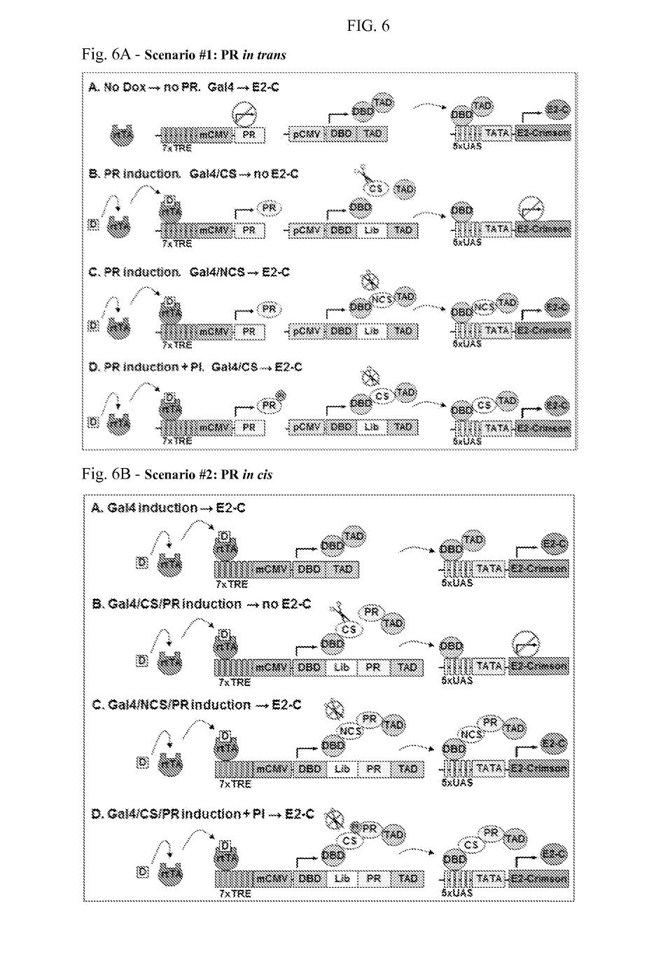

The main differences between the exemplary assay described in FIG. 1 and the alternative embodiments include: the exchange of GFP with the far-red fluorescent protein E2-Crimson for better detection, and the introduction of a library of potential cleavable sites between the Gal4 domains. Alternative embodiments (two scenarios) are based on how PR is supplied in order to ensure the utility of the assay to all manner of viral proteases. In one embodiment, or Scenario #1 (FIG. 6, scenario #1), PR is supplied in trans through an independent vector. While PR is expressed under an inducible promoter, the Gal4/library fusion is constitutively expressed. In one embodiment, or scenario #2 (FIG. 6, scenario #2) PR is supplied in cis as part of the Gal4 fusion construct. In one embodiment, the entire Gal4/library/PR fusion is expressed under an inducible promoter. In one embodiment, there is stable expression of the assay elements utilizing retroviral vectors. In alternative embodiments, the assays are inducible systems for the expression of PR, whether in cis or in trans. In alternative embodiments, the assays replace the HIV-1 PR or the PR cleavage sites adjacent to PR with segments from a cDNA library.

FIG. 6: Overview of this exemplary assay:

FIG. 6A: Scenario #1: PR supplied in trans. A. Wild type Gal4 as control. E2-C expression is expected from the reporter construct. B. In the presence of Dox (the “D” box), PR expression is induced. In the presence of a cleavable site, the Gal4 domains are separated, and thus no E2-C expression is expected. C. As in B but in the absence of a cleavable site. Gal4 domains remain intact, resulting in the induction of E2-C expression. D. As in B but in the presence of PI (orange circles). Gal4 fusion remains intact, resulting in the induction of E2-C expression.

FIG. 6B: Scenario #2: PR supplied in cis. A. Wild type Gal4 as control. In the presence of Dox (the “D” box), E2-C expression is expected from the reporter construct. B. PR fusion expression is induced. In the presence of a cleavable site, the Gal4 domains are separated, and thus no E2-C expression is expected. C. As in B but in the absence of a cleavable site. Gal4 domains remain intact, resulting in the induction of E2-C expression. D. As in B but in the presence of PI (orange circles). Gal4 fusion remains intact, resulting in the induction of E2-C expression. (CS: cleavable site, NCS: non-cleavable site. E2-C: E2-Crimson, Lib: Library).

In alternative embodiments, the assays of the invention are adapted to substrates of PR: an exemplary assay is adapted to putative substrate/s of PR rather than PR itself. Known PR substrates are used as controls to corroborate the robustness and usability of the assay for the search of novel candidate substrates.

In alternative embodiments, two libraries are constructed from the cDNA of SupT1 T-cells based on the average size of their segments: an approximately 500 bp and an approximately 2000 bp-long library. The libraries can be introduced into the retroviral Gal4-based vector for stable expression in mammalian cells.

Rationale: In the recent report by Jager et al, 2012, an interactome approach was undertaken through affinity purification coupled with mass spectrometry, revealing the physical interactions of all 18 HIV-1 proteins and polyproteins with host proteins. Among those, at least 50 cellular proteins were shown to bind PR. As mentioned above, subunit D of the translational initiation complex eIF3 was shown to be cleaved by PR. HIV-1 PR is known to have autocatalytic activity as well as catalytic activity. One exemplary assay monitors the autocatalytic activity of PR in cis. The fact that PR is known to act on all cleavage sites of the HIV-1 proteome (except in Envelope) and many known host targets, assures us that the assay will work when PR is supplied in trans as well. As we will perform a second independent screen in parallel with PR supplied in cis as a fusion with the putative substrate, we will increase the chance of revealing novel substrates. The screening process for the search of cellular targets of HIV-1 PR should be straightforward, as non-cleaved targets should result in green fluorescence and cleaved targets would show lack of or drastic decrease in fluorescence. Moreover, the fact that several cellular proteins have been shown to be recognized and cleaved by HIV-1 PR, implies that other targets are yet to be found. The cell-based nature of the assay mimics the natural milieu for PR activity, especially considering that many of the PR targets might be recognized and cleaved only in the presence of other factors or co-factors, making the cell-based assay the perfect tool for their discovery. Moreover, HIV-1 has been recently shown to use alternative mechanisms to become resistant to PIs by changing the substrate rather than PR, and potentially substrate specificity, further corroborating the importance of discovering PR substrates in an appropriate environment. As one assay embodiment was developed for wild-type HIV-1 PR, its adaptation to the substrates of PR rather than PR itself is straightforward. This alternative exemplary assay of the invention is based on the Gal4 yeast transcription factor. The property of the DBD and TAD domains of Gal4 serves as the template for this exemplary assay, where reporter gene expression is inversely proportional to the ability of PR to recognize and cleave a substrate placed between the Gal4 domains.

Comparison of PR Supplied in Trans and in Cis:

One exemplary assay relies on the ability of PR to cleave itself out of the fusion in cis, (see FIG. 2, Gal4-based constructs), although it does not eliminate the possibility of cleavage in trans through dimerization. In order to adapt the assay to the search of PR substrates instead of PR activity, it is critical to ensure maximum activity of PR. We first corroborate PR activity with known substrates introduced in the context of the Gal4 fusion and compare the activity of PR when supplied in trans and in cis (scenarios #1 and #2 respectively).

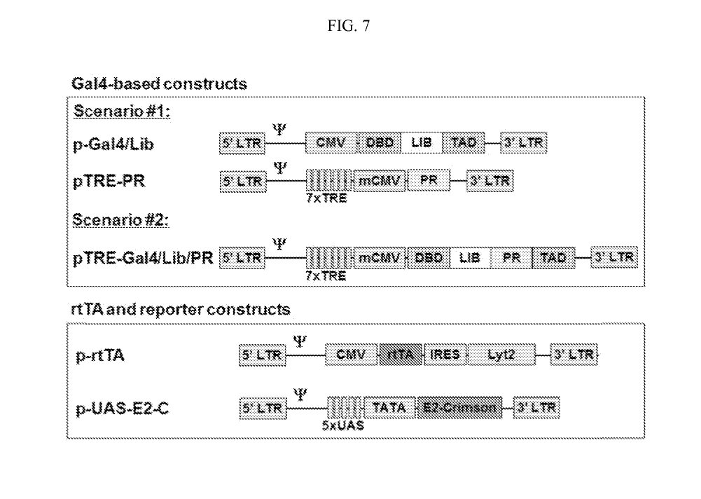

One exemplary assay construct included the two recognition/cleavage sites flanking PR, which was in the context of the Gal4 fusion. For an alternative embodiment, an alternative assay, PR will be supplied in trans in an independent plasmid (FIG. 7, Gal4-based constructs in scenario #1), or in cis as part of the fusion (FIG. 7, Gal4-based construct in scenario #2). For the system in trans PR will be cloned in an independent plasmid under the TRE inducible promoter, as in the original vector. This will allow for the induction of PR expression when needed by supplying Dox. In this system, the cDNA segments library will be cloned between the DBD and TAD domains of Gal4. For the system in cis, PR will be kept between the Gal4 domains, but the original cleavage sites flanking PR will be deleted. Instead, the library will be introduced as a fusion between the DBD Gal4 domain and PR. The original rtTA construct contained an IRES-mCherry cassette, which was further substituted with an IRES-Lyt2 cassette (mouse-CD8) allowing corroboration of the presence of rtTA by flow cytometry rather than red fluorescence. It is worth mentioning that the presence of rtTA is indirectly proved by the successful induction of the system following addition of Dox. The exchange between mCherry (FIG. 3, rtTA construct) and Lyt2 was needed in order to avoid possible interference between mCherry and the far red fluorescent protein E2-Crimson of the new reporter construct where E2-Crimson replaces GFP (compare reporter construct of FIGS. 3 and 4). The following constructs represent the alternative embodiments, or elements, of the exemplary assay:

As controls, two known cleavage sites from the HIV-1 proteome (PR/RT and RT/IN boundaries) and a newly discovered cellular target; Eif3d, are introduced. The DNA of Eif3d along with a non-cleavable version of Eif3d was kindly supplied by Charles Craik, University of California San Francisco.

Rationale: The fusion of PR to the Gal4 DNA-binding and trans-activation domains is by no means intended to mimic the physiological environment, but as demonstrated in our previous publication with HIV-1 PR, it clearly allows the assessment of viral PR activity and its inhibition by all the FDA-approved inhibitors. PR is known to be active in the cytosolic environment, whether in the context of the cell or inside the viral capsid; cleavage in the context of the Gal4 fusion most likely also occurs in the cytosol.

While this exemplary assay facilitates the discovery of PR substrates, additional assays are performed to corroborate the nature of putative hits as true cleavable substrates. While the HIV-1 PR is known to be active without the need for cofactors, this is not the case for many proteases of other viruses, especially Flaviviridae. The HCV NS3 protease essential for polyprotein processing, works in complex with NS2 and/or NS4A cofactors. NS2, an accessory protein and an integral membrane protein targeted to the endoplasmic reticulum, is an autoprotease responsible for cleaving the NS2/3 boundary. Similarly, Dengue virus, West Nile virus or Yellow Fever virus require the NS2B factor for full activity. The DenV NS3 serine protease activity is dependent on the presence of the NS2B cofactor as it wraps around the NS3 domain, becoming part of the active site and thus required for full activity. Thus, two scenarios facilitate the utility of exemplary assays of the invention for both kind of proteases; those that have full activity on their own and those that depend on cofactors. The alternative embodiment (system) where protease is supplied in trans, will allow expression of a protease by itself or as a complex with its cofactor/s. For those proteases that depend on co-factors, the alternative embodiment (system) in cis requires the deletion of the cleavage site within the protease sequence. Otherwise the Gal4 fusion can be destroyed independently of the presence or absence of a putative substrate downstream DBD and upstream protease. Additionally, it will be possible to replace the cleavage site between protease and co-factor with a non-cleavable but flexible linker such as polyglycine-serine, without affecting protease activity.

FIG. 7 schematically illustrates retroviral/lentiviral constructs utilized in this exemplary assay. Constructs utilized to generate infectious particles for the transduction of SupT1 cells with the various assay elements including the Gal4-based vectors: Gal4/Lib and PR for scenario #1 and Gal/Lib/PR for scenario #2, and rtTA, and E2-Crimson reporter constructs. (LTR: Long Terminal Repeat, packaging signal, Lib: library.

Construction of cDNA Fragment Libraries:

As source for cellular proteins, two libraries are constructed from the cDNA of SupT1 T-cells. They differ in the average size of their segments: about 500 bp and about 2000 bp-long library. The libraries are engineered as part of the Gal4 retroviral vector and are stably expressed in mammalian cells.

Construction of the Libraries:

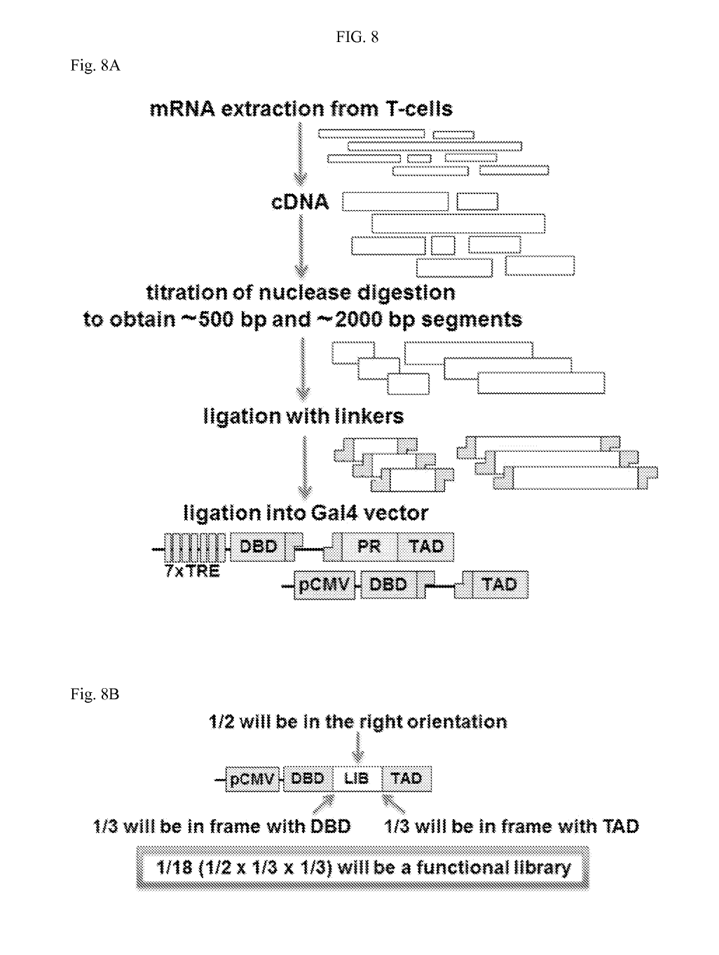

In order to obtain a cDNA fragment library, mRNA from SupT1 cells are extracted. For that purpose, cells are lysed with lysis/binding buffer and subjected to DYNABEADS OLIGO (DT)25™ (Invitrogen). The mRNA will be then eluted and fragmented using NEBNext® RNase III fragmentation module to generate fragments in the range of about 500 and about 2000 nucleotides. This is similar to obtaining expressed sequence tags or ESTs but here we calibrate digestion to obtain two size ranges (see below). The resulting fragments will undergo reverse transcription to create a RNA/cDNA hybrid, which, following RNase H treatment, and subsequent addition of polymerase I will result in double stranded (ds) cDNA. cDNA's ends will be repaired to create “blunt ends” using the End-it™ DNA End-Repair Kit and ligated to specific linkers that will allow easy cloning between the DBD and TAD region of the Gal4-based expression vectors. FIG. 8A describes the outline for the library construction.

Complexity of the Library:

Due to physical constraints of ligation of a blunt cDNA library within the Gal4 fusion, only a percentage of the library is expected to be functional, i.e., to produce a protein in frame throughout both the DBD and TAD domains of Gal4. FIG. 8B illustrates the calculation of the percentage of the functional library within the overall library complexity.

Rationale: Assuming 25,000 genes in the human genome, a theoretical library of cDNA with a maximum representation of different genes should include at least ten of each in average in order to account for differential expression; that is 250,000 clones. In order to ensure functional representation of each gene, the complexity of the library should be 18×2.5×10⁵, or 4.5×10⁶ individual clones. Our expertise with the production of random peptide libraries, which range in complexity between 1×10⁶ to 1×10⁷ individual clones, reassures us of the feasibility of obtaining a high complexity library, and thus a high representation of independent genes. By mixing 500 and 2000 bp-long segments we hope to increase the chances of introducing both shorter and longer transcripts, and thus increase the chances of obtaining a better representation of proteins as intact as possible. The nature of the library for fusion implies losing some information, as some segments may not retain the three-dimensional structure that may be needed for PR recognition and cleavage. In one embodiment, about 500 and about 2,000 bp-long cDNA, representing about 165 and about 665 amino-acid-long protein fragments, will include large protein fragments, some of which will include close to complete proteins, thus increasing the probability of obtaining near wild-type conformations. In one embodiment, the library is fused to a fluorescent protein to ensure expression of the full fusion (DBD through TAD) to allow selection of cells that express in-frame peptides. That is not necessary as the screen itself overcomes the need for such fluorescent fusion.

FIG. 8A: Outline of library construction. mRNA is extracted from SupT1 T-cells and used as template for cDNA. cDNA is digested with nuclease to obtain a library of ˜500 bp-long segments and a library of 2000 bp-long fragments. Following ligation with linkers, the libraries are introduced into the Gal4-based vectors.

Screen for Putative Novel PR Substrates:

In one embodiment, once the libraries are constructed and introduced into SupT1 cells through retroviral technology, cells are utilized for the screening of cellular PR substrates. Below are described two alternative embodiments (two different scenarios): The first embodiment is a negative screen and is based on the expression of PR in trans, in an independent inducible construct. The second embodiment is a positive screen and is based on the inducible expression of PR in cis, as part the Gal4 fusion. Whether embodiment (scenario) #1, embodiment (scenario) #2, or both are chosen for a particular purpose may vary. In one embodiment, if PR works well in trans and allows clear discrimination between cleaved and non-cleaved known targets, scenario #1 can be chosen. In one embodiment, if better discrimination is obtained with PR in cis as part of the Gal4 fusion, scenario #2 is chosen.

In alternative embodiments, while two independent screens can be compared, they are not incompatible and can be performed concomitantly. Two unrelated screens can further corroborate the results obtained from each one independently. Potential hits can be further validated to corroborate their nature as true PR-cleavable host proteins. It is important to mention that HIV-1 PR, utilized here as proof of principle, is active by itself and does not require co-factors. In alternative embodiments, for proteases that do require cofactors, both protease and cofactor should be supplied I or, when supplied in cis, a non-cleavable protease-cofactor fusion should be used instead Such as the one described above, with a polyglycine/serine linker.

Screening Process—

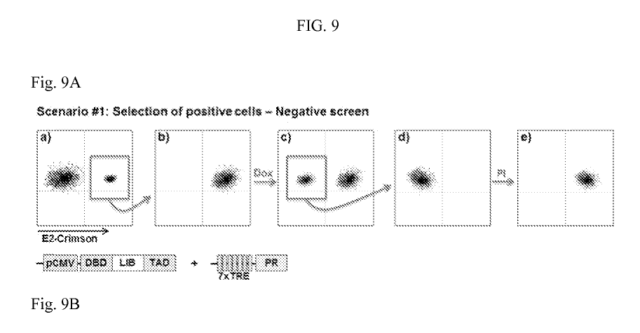

Exemplary embodiment (Scenario) #1: are performed only if PR is supplied in trans in an inducible manner via a second plasmid (FIG. 9). In this scenario, the Gal4/library fusion construct is constitutively expressed in cells bearing the inducible PR construct. While Dox is absent, the system will behave as if PR is not present. The Gal4 fusions will remain intact unless they are cleaved by other proteases in the cell. As such, they will be able to activate the expression of the E2-Crimson fluorescent gene. In one embodiment, fluorescent cells (E2-Crimson positive) are sorted out (to enrich for the population of cells expressing Gal4/library in-frame intact fusions which include both Gal4 DBD and TAD (panel a). Importantly, substrates that are cleaved by other proteases other than PR will be destroyed and as they will not be able to activate E2-Crimson expression, and will thus be lost prior to the sort. This embodiment (scenario) will thus lead to substrates specifically cleaved by PR from the very start of the screen. Once E2-Crimson positive cells are enriched (panel b), PR expression will be induced with the addition of Dox. A small population of cells is expected to lose fluorescence (panel c). This population will mostly include cells expressing a target for PR, which should destroy the Gal4/library fusion protein. By enriching this negative population (panel d) one expects to enrich for cells expressing putative PR substrates. Addition of PI (100.M Indinavir) should pinpoint the cells that express a PR substrate, as PR, although present, should be inactive, resulting in de novo synthesis of intact Gal4/library fusion able to activate E2-Crimson gene (panel d). Individual cells can be sorted into wells of 96-well plates and amplify to obtain clonal populations. Clonal populations can be further used to rescue the sequence of the potential substrate (see below).

FIG. 9 illustrates a cell sorting/FACS selection process and screening for embodiment (scenario) #1, where PR is supplied in trans in an inducible manner. Before induction, cells expressing the Gal4/library fusion and thus E2-Crimson-positive, FIG. 9A(a) are sorted out FIG. 9A(b). Following Dox induction, E2-Crimson negative cells FIG. 9A(c) are sorted out FIG. 9A(d) and corroborated with PR inhibitor (PI) FIG. 9A(e). (Flow cytometry plots are made-up for the sake of illustration only. Dox: Doxycycline, PI: protease inhibitor. See text for details). The inducible PR construct used in this exemplary cell sorting/FACS selection process is illustrated in FIG. 9B.

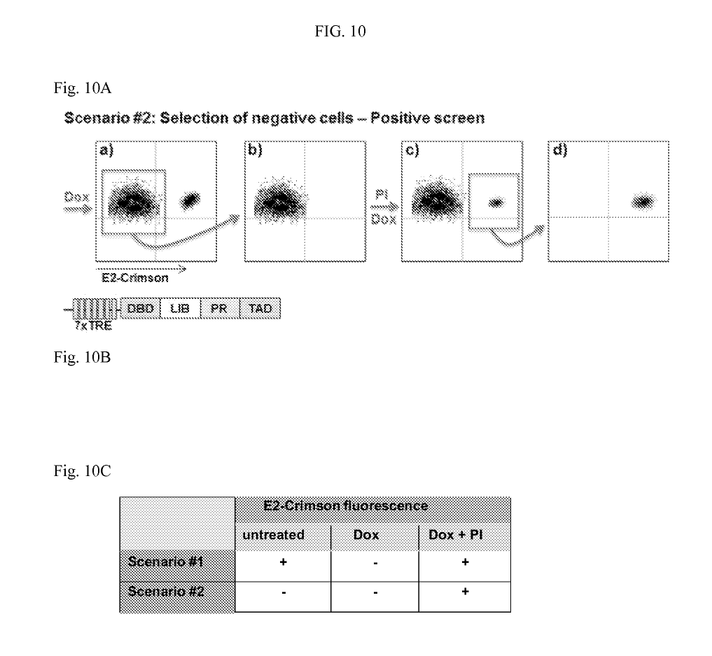

An exemplary assay/screening process of the invention, as illustrated in FIG. 10, is an embodiment (Scenario #2) performed by supplying PR in cis within the context of the Gal4 fusion in an inducible manner. The screen starts with the addition of Dox. In alternative embodiments, cells can be non-fluorescent and not contain a Gal4/library fusion protein at all, or they can express a fusion protein cleaved by HIV-1 PR or cellular proteases. Cells expressing a non-cleaved fusion can be fluorescent (FIG. 10A panel a) as they will be able to activate the E2-Crimson reporter. This population is not of interest in this screen. In alternative embodiments, the non-fluorescent population is sorted and expanded (FIG. 10A panel b). In alternative embodiments, cells are then treated again with Dox, but this time in the presence of PI (100.M Indinavir). We have shown that Indinavir at this concentration, and PR as part of a fusion, are not toxic at least for the time frame required for the screen. Only the subpopulation of cells that express a substrate specific for PR should turn fluorescent and thus be E2-Crimson positive (FIG. 10A panel c). In alternative embodiments, this population is sorted and amplified (FIG. 10A panel d) and used to obtain individual clonal populations by growing individual cells in a 96-well format.

Selection of Clones:

In one embodiment, whether clones are obtained from the negative screen described in the exemplary embodiment Scenario #1, or the positive screen described in the embodiment Scenario #2, only those that behave as expected are analyzed further. To corroborate their expected behavior, they can be left untreated for a period of one to two weeks and then treated, as illustrated in FIG. 10C.

Caveats: Corroboration of expected clonal behavior as described here is based on the availability of protease inhibitors. While this is clearly possible for HIV-1, it will probably not be the case for many other viral proteases. When no inhibitors are available, an additional step should be required, where each individual rescued hit should be expressed, in parallel, in the presence of the protease of interest, and also in the presence of a non-active mutant version of the protease.

Rescue of Cellular Substrates:

In alternative embodiments, clones that behave as required (see the above-mentioned table) are used to purify their genomic DNA, which can serve as template for PCR amplification of the Gal4/library fusion DNA sequence. The sequences obtained, which can represent only gene segments, can be compared to the human DNA database to retrieve the sequence of the complete genes. Follow-up of each hit, including the introduction of the entire protein in the context of the assay, can be performed to corroborate whether their product is indeed cleaved by HIV-1 PR. E2-Crimson fluorescence should be observed only in the presence of a PI if the putative substrates are indeed cleaved by PR.

As discussed above, FIG. 10 illustrates the exemplary selection process and screening assay embodiment of the invention (Scenario #2), where PR is supplied in cis as part of the Gal4-fusion. Following Dox induction, E2-Crimson negative cells, as illustrated in FIG. 10A(a) are sorted out, as illustrated in FIG. 10A (b); and following addition of Dox and PI, E2-Crimson positive cells, as illustrated in FIG. 10A(c), are sorted out, as illustrated in FIG. 10A(d). Flow cytometry plots are made-up for the sake of illustration only. Dox: Doxycycline, PI: protease inhibitor.

Validation of Potential Hits:

In alternative embodiments, obtained potential hits are further validated to corroborate their nature as true PR-cleavable host proteins. We have previously performed biochemical assays where PR-based processing of the Gag-Pol precursor was analyzed in the absence or presence of PI. The presence of CA (capsid), which accumulates only in the absence of PI, was corroborated with antibodies by Western blot (see preliminary data, FIG. 5). Similar experiments can be performed with each of the potential hits fused to the FLAG tag and analyzed for size in the presence of PR prior and following the addition of PI.

Caveats: In Embodiment (Scenario) #1, PR is supplied through an independent inducible plasmid. Some E2-Crimson background is expected as the Gal4 fusion, which is constitutively expressed, is present prior to its cleavage by PR. This will certainly occur even if the host protein/s serve as true PR substrates. In this case, we expect to see a decrease in E2-Crimson fluorescence rather than an all-or-none fluorescence following the addition of Dox to activate PR expression. It is possible that following the addition of Dox it will be necessary to proceed for several days before analysis to allow pre-existing E2-Crimson to be degraded and ensure a decrease in fluorescence background. If necessary, a degron sequence can be added to E2-Crimson to ensure faster turnover. If the signal-to-noise ratio is not high enough to perform the negative screen described in scenario #1, the Gal4 fusion can be transferred to an inducible system, and perform a screen similar to scenario #2 with PR in trans. In order to increase the stringency of the screen, the validation process described above (selection of clones), which can be carried out with the final clonal populations, can be performed with sorted populations of cells at any time throughout the screen. Because of the nature of the screen, an all-or-none type of response might not be expected. Instead, a decrease in fluorescence and recovery in the presence of inhibitor or in the presence of a mutated non-active version of the protease might be observed.

Production of Retroviral Particles:

For the production of MLV-based viral particles, Phoenix-GP packaging cell line (kindly provided by Garry Nolan, Stanford University) is transfected with retroviral vectors. For the production of HIV-based virus particles, 293T cells are transfected with pH-GFP transfer vector, pCI-VSVg, and pCMV . . . 8.2 (Didier Trono, EPFL, Switzerland). In each case, viral supernatant is collected at 48 hours post-transfection. Viral supernatant is used to transduce SupT1 cells by centrifugation at 1500×g, at 32° C. for 80 minutes. Cells harboring reporter and rtTA plasmids are further transduced with viral particles carrying Gal4 control, Gal4-PR substrate fusions and/or PR expression vector.

Flow Cytometry and Sorting:

Flow cytometry was performed on a BD FACSARIA™ (FACSAria™) (SDSU FACS core facility) with 488 nm and 633 nm lasers. Data was collected using FACSDiva 6.1.1™ software and analyzed by FlowJo™.

Transient Expression Corroborates Usability of Exemplary Assays of the Invention with PR Supplied in Trans:

To corroborate the usability of the assay for substrate discovery, and prove whether PR can be supplied in trans, it was critical to prove PR activity with known viral and host substrates. For that purpose, the well-characterized PR/RT and RT/IN HIV-1 boundaries were introduced between the Gal4 domains.

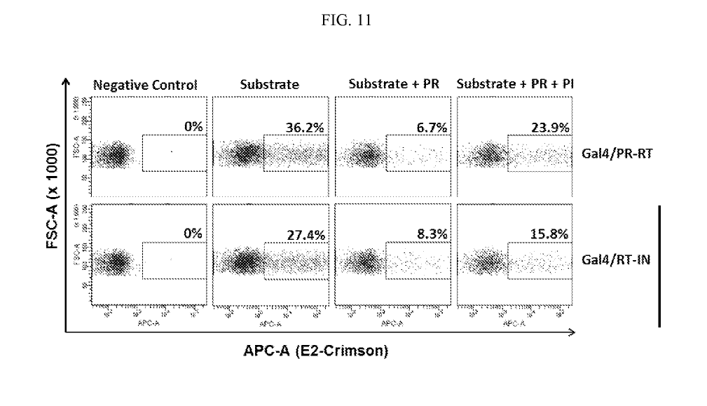

FIG. 11 illustrates the results of a FACS/flow cytometry analysis, a transient experiment with HEK 293T cells; in particular, a flow cytometry analysis of cells transfected with E2-C reporter and rtTA alone (negative control), or in addition to PR/RT (upper panels) or RT/IN (lower panels) substrate, substrate with PR, or substrate with PR and Darunavir as PI. The FACS/flow cytometry analysis data illustrated in FIG. 11 shows no background with rtTA and reporter, and a drastic induction of E2-C fluorescence reporter when Gal4-PR/RT or RT/IN were added. Importantly, PR when supplied as a GFP/PR fusion, an 80% or 60% decrease in fluorescence was observed respectively. This was strongly inhibited in the presence of the PI Darunavir, further demonstrating that the reduction in fluorescence was due to PR activity.

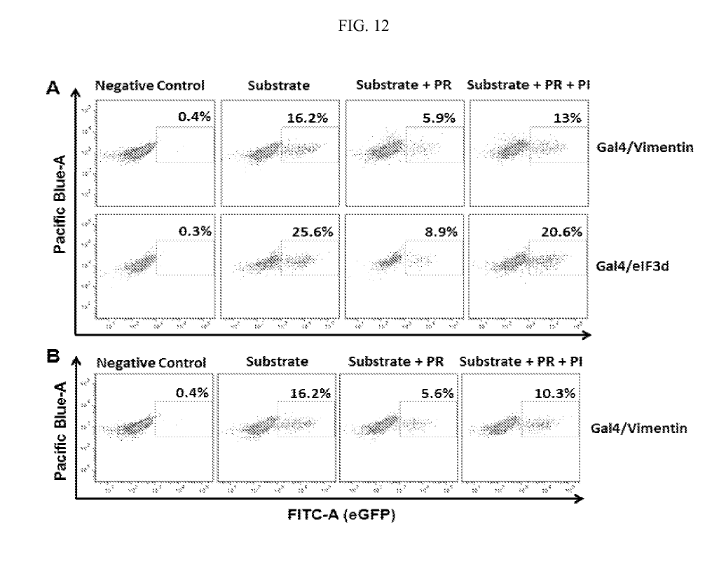

When the experiment was performed with the inducible expression of the newly discovered cellular target eIF3d, (kindly supplied by Charles Craik from UCSF) and vimentin (PCR rescued from cDNA) (FIG. 12A) a similar result was obtained. In this case, mCherry/PR was used to show versatility.

In order to further demonstrate that a more biologically relevant source of PR can be used, the same experiment was repeated with Gag-Pol and Gag-PR. Results obtained for vimentin with Gag-PR, which were similar to those with Gag-Pol are shown in FIG. 12B.

In summary, FIG. 12 illustrates results of the transfection of PR with HEK 293T cells stably expressing rtTA and cellular substrates. FIG. 12A: Five hours following transfection with mCherry/PR, Darunavir as PI was added; five hours later, Dox was added to activate Gal4/vimentin or Gal4/eIF3d substrate expression. FIG. 12B. As FIG. 12A for Gal4/vimentin, but with Gag-PR as source of PR.

A number of embodiments of the invention have been described. Nevertheless, it will be understood that various modifications may be made without departing from the spirit and scope of the invention. Accordingly, other embodiments are within the scope of the following claims.

1: An isolated, synthetic or recombinant polypeptide or peptide comprising or consisting of
an amino acid sequence comprising or consisting of the formula:
(SEQ ID NO: 1)
Thr Arg Arg Val Ala His Asn Ser Glu (TRRVAHNSE);
[[or]]
(SEQ ID NO: 2)
Trp Arg Gly Ala Ala Met Val Arg Gly (WRGAAMVRG);
or
(c) a combination of (both) SEQ ID NO:1 and SEQ ID NO:2; or
(b) a peptidomimetic or a bioisostere of the isolated, synthetic or recombinant polypeptide or peptide of (a).

2: A chimeric protein comprising an isolated, synthetic or recombinant polypeptide or peptide of claim 1, wherein the chimeric protein comprises:
(A)
(a)(i) a first domain comprising an isolated, synthetic or recombinant polypeptide or peptide of claim 1; and, (ii) at least a second domain or moiety;
(b) the chimeric protein of (a), wherein the chimeric protein comprises a recombinant fusion protein; or
(c) the chimeric protein of (a) or (b), wherein the second domain or moiety comprises a targeting agent;
(B) the chimeric protein of (A), wherein the targeting agent comprises an antibody Fc domain or an antibody that binds to an Fc receptor, or a chimeric protein comprising two or more antibody Fc domains;
(C) the chimeric protein of (A) or (B), wherein the at least second domain or moiety comprises an Fc domain, a protein C, an antibacterial or bacteriostatic peptide or protein, an antibiotic, a cytokine, an immunoregulatory agent, an anti-inflammatory agent, a complement activating agent, a carbohydrate-binding domain or a combination thereof;
(D) the chimeric protein of any of (A) to (C), wherein the chimeric protein comprises a recombinant, peptidomimetic or synthetic protein; or
(E) the chimeric protein of any of (A) to (D), wherein the first domain is joined to the second domain or moiety by a chemical linking agent.

3: A composition comprising:
(a) a first composition comprising the isolated, synthetic or recombinant polypeptide or peptide of claim 1, and a second composition;
(b) the composition of (a), wherein the second composition comprises a liquid, a lipid or a powder;
(c) the composition of (a) or (b) formulated as a protein preparation, wherein optionally the protein preparation comprises a liquid, a slurry, a powder, a spray, a suspension, a lyophilized composition/formulation, a solid, geltab, pill, implant, a gel; or a pharmaceutical formulation, a food or a feed or a supplement thereof; or
(d) the composition of (a) or (b) immobilized on or inside a cell, a vesicle, a liposome, a film, a membrane, a metal, a resin, a polymer, a ceramic, a glass, a microelectrode, a graphitic particle, a bead, a gel, a plate, an array, a capillary tube, a crystal, a tablet, a pill, a capsule, a powder, an agglomerate, a surface, or a porous structure.

4: A liposome comprising:
(a) the isolated, synthetic or recombinant polypeptide or peptide of claim 1; or
(b) the liposome of (a), wherein the liposome is formulated with a pharmaceutically acceptable excipient.

5: A pharmaceutical composition comprising: the isolated, synthetic or recombinant polypeptide or peptide of claim 1; and, a pharmaceutically acceptable excipient.

6: An inhalant or spray formulation comprising: the isolated, synthetic or recombinant polypeptide or peptide of claim 1; and, a pharmaceutically acceptable excipient.

7: A parenteral or enteral formulation comprising: the isolated, synthetic or recombinant polypeptide or peptide of claim 1, or any combination thereof; and, a pharmaceutically acceptable excipient.

8. (canceled)

9: An isolated, synthetic or recombinant nucleic acid encoding the isolated, synthetic or recombinant polypeptide or peptide of claim 1.

10: An expression cassette, vector, cloning vehicle, expression vector, cloning vector:
(a) comprising the nucleic acid of claim 9, or having contained therein a nucleic acid of claim 9;
(b) the expression cassette, vector, cloning vehicle, expression vector, cloning vector of (a), wherein the cloning vehicle comprises or is a viral vector, a plasmid, a phage, a phagemid, a cosmid, a fosmid, a bacteriophage or an artificial chromosome, and optionally the viral vector comprises an adenovirus vector, a retroviral vectors or an adeno-associated viral vector, and optionally the expression cassette, vector, cloning vehicle, expression vector, cloning vector comprises or is contained in a bacterial artificial chromosome (BAC), a plasmid, a bacteriophage P1-derived vector (PAC), a yeast artificial chromosome (YAC), a mammalian artificial chromosome (MAC).

11. (canceled)

12: A cell, a transformed cell or a host cell comprising:
(a) the nucleic acid of claim 9;
(b) the transformed cell or host cell of (a), wherein the cell is a bacterial cell, a mammalian cell, a fungal cell, a yeast cell, an insect cell or a plant cell; and optionally the bacterial cell is any species within the genera Escherichia, Bacillus, Streptomyces, Salmonella, Pseudomonas or Staphylococcus, or Escherichia coli, Lactococcus lactis, Bacillus subtilis, Bacillus cereus, Salmonella typhimurium or Pseudomonas fluorescens.

13. (canceled)

14: A method for treating, ameliorating, slowing the progress of, reversing, or preventing a retroviral infection, or an HIV-1 infection, or AIDS, in an individual in need thereof, comprising:
(A)
(a) providing the isolated, synthetic or recombinant polypeptide or peptide of claim 1; and
(b) administering an effective amount of (a) to the individual, thereby treating, ameliorating, slowing the progress of, reversing, or preventing a retroviral infection, or an HIV-1 infection, or AIDS; or,
(B) the method of (A), further comprising co-administration of at least one anti-retroviral or anti-HIV-1 drug, composition, therapy or diet.

15: An isolated, recombinant or synthetic nucleic acid comprising or consisting of a construct as set forth in FIG. 2, FIG. 6, FIG. 7, FIG. 9B or FIG. 10B.

16: A vector, expression cassette, cosmid or plasmid comprising or having contained therein the isolated, recombinant or synthetic nucleic acid of claim 15.

17: A cell, a transformed cell or a host cell comprising,
or having contained therein, the isolated, recombinant or synthetic nucleic acid of claim 15.

18: A chimeric polypeptide encoded by a nucleic acid comprising or consisting of a construct as set forth in FIG. 2, FIG. 6, FIG. 7, FIG. 9B or FIG. 10B.

19: A cell, a transformed cell or a host cell comprising, or having contained therein, the chimeric polypeptide of claim 18.

20: A chimeric or hybrid polypeptide comprising or consisting of: the polypeptide encoded by the nucleic acid of claim 15, wherein the protein comprises a synthetic protein or peptide, recombinant protein or peptide, a peptidomimetic or a combination thereof.

21: A cell, a transformed cell or a host cell comprising the isolated, recombinant or synthetic nucleic acid of claim 15, wherein optionally the cell is a mammalian or a human cell, wherein optionally the cell is a hepatocyte, or a lymphocyte or a T cell, or a CD4+ T cell.

22: A cell-based method, for:
(i) monitoring the activity of an enzyme, or an HIV-1 protease (PR) enzyme, or
(ii) for screening for a substrate of the enzyme or, the HIV-1 protease (PR) enzyme, comprising:
(1)
(a) providing:
(A)
(i) a vector, expression cassette, cosmid or plasmid of claim 10;
(ii) a pairing or grouping of constructs as set forth in FIG. 1 or FIG. 6, or a construct as set forth in FIG. 2, FIG. 6, FIG. 7, FIG. 9B or FIG. 10B; or
(iii) a pairing or grouping of constructs comprising:
(1) a construct wherein an enzyme, or a PR HIV-1, is fused between a DNA-binding domain (DBD) and a transactivation domain (TAD) of a transcription factor (optionally a Gal4 transcription factor), wherein the enzyme or PR fused within the transcription factor auto-catalytically can cleave itself resulting in two non-functional transcription factor domains, and
(2) a construct encoding a detectable moiety or protein operatively linked to a transcription factor (optionally a promoter) which is only active when activated by the transactivation domain (TAD) of a transcription factor (optionally a Gal4 transcription factor) of (1),
and optionally the detectable moiety or protein comprises a fluorescent protein, a green fluorescent protein (GFP), a far-red fluorescent protein, an E2 crimson fluorescent protein, or equivalents thereof; and,
(B) a cell comprising an environment capable of supporting the expression of the chimeric (hybrid) protein by the nucleic acid;
(b) inserting or transfecting or infecting the nucleic acid, vector, recombinant virus, cloning vehicle, expression cassette, cosmid or plasmid of (a) into the cell;
(c) contacting the cell with a putative (test) enzyme inhibitor or activator,
wherein optionally the enzyme is provided in cis or in trans,
wherein optionally an enzyme inhibitor or activator is added to the cell before, during and/or after inserting (transfecting) the nucleic acid, vector, recombinant virus, cloning vehicle, expression cassette, cosmid or plasmid of (a) into the cell and/or expressing the chimeric protein encoded by a nucleic acid of (a) in the cell,
and optionally the cell-based method further comprises a negative, positive and/or alternative control set of cells into which the nucleic acid, vector, recombinant virus, cloning vehicle, expression cassette, cosmid or plasmid of (a) also has been inserted (or transfected) and expresses the chimeric protein encoded by a nucleic acid of (a), but the negative control set of cells is not exposed to an enzyme (optionally an HIV-1 protease (PR) enzyme) inhibitor or activator, or is exposed to a different or known enzyme substrate (optionally an HIV-1 protease (PR) enzyme substrate), or a different amount of substrate or inhibitor or activator, or a positive control wherein the cells are exposed to a known substrate of the enzyme; and
(d) determining whether a putative (test) enzyme substrate is sufficiently cleaved by the enzyme or an enzymatically active fragment thereof,
optionally by measuring the ability of the enzyme inhibitor to partially or completely inhibit cleavage of the putative (test) enzyme substrate; or
determining whether the putative inhibitor of the enzyme inhibits the enzyme to result in sufficient active transcription factor (optionally Gal4 transcription factor) to be produced to activate production of the detectable moiety or protein, which optionally comprises a fluorescent protein, a green fluorescent protein (GFP), a far-red fluorescent protein, an E2 crimson fluorescent protein, or equivalents thereof (if the enzyme is inhibited partially or completely, it does not cleave the transcription factor, which can then activate production of the detectable moiety or protein),
wherein optionally the fluorescent protein is detected by a fluorescent activated cell sorter (FACS);
(2) the cell-based method of (1), further comprising measuring the ability of the enzyme to partially or completely cleave the enzyme substrate comprises detecting and/or measuring the amount of tag or detection moiety, or “scaffold”, on the cell surface;
(3) the cell-based method of (1) or (2), wherein the cell is a hepatocyte, a lymphocyte or a T cell, or a CD4+ T cell, or a human cell;
(4) the cell-based method of any of (1) to (3), further comprising running a negative control comprising dividing the plurality of the cells co-expressing a nucleic acid of (a) in the cell and not adding the substrate to be screened (the putative (test) substrate) to one of the divided cell samples;
(5) the cell-based method of any of (1) to (4), further comprising running a positive control comprising dividing the plurality of the cells co-expressing the nucleic acid of (a) in the cell and adding a known substrate of the enzyme, or a known substrate of a furin enzyme, a calcium-dependent protein convertase enzyme, prohormone convertase-1 (PC1) enzyme, or an enzyme from a member of the subtilisin/kexin family of proprotein convertases, to one of the divided cell samples; or
(6) the cell-based method of any of (1) to (5), wherein:
(a) the transcriptional regulatory unit comprises a promoter, an inducible promoter or a constitutive promoter;
(b) the cell is a mammalian cell, a monkey cell or a human cell, or the cell is a mammalian cell, a monkey cell or a human cell, or a hepatocyte, a lymphocyte, or a T cell, or a CD4− or CD8-expressing cell, and optionally the cells are genetically bar-coded;
(c) the detectable moiety or protein, which optionally comprises a fluorescent protein, a green fluorescent protein (GFP), a far-red fluorescent protein, an E2 crimson fluorescent protein, or equivalents thereof; or the tag or detection moiety, or “scaffold”; is detected or measured in the cell or on the cell surface by a high throughput screen, a plate reader, a flow cytometry or a microscope visualization;
(d) the (putative) inhibitor of the enzyme comprises a small molecule, a nucleic acid, a polypeptide or peptide, a peptidomimetic, a polysaccharide and/or a lipid; or
(e) the substrate to be screened is a member of a library of substrates to be screened, or a member of a random peptide or polypeptide library; or
(6) the cell-based method of any of (1) to (6), wherein two or more, or a plurality of, enzymes are screened in the same cell, wherein optionally they are variants of the same enzyme or protease, or different enzymes or proteases, or a combination thereof.

23: The method of claim 22, wherein:
(a) the enzyme or the HIV-1 protease (PR) enzyme is supplied in trans in an inducible manner;
and optionally, before induction, cells expressing the Gal4/library fusion and are positive for a detectable moiety are sorted out,
and optionally the detectable moiety comprises a fluorescent protein, the GFP, or the E2-Crimson;
(b) the enzyme is supplied in cis as part of the Gal4-fusion; and optionally, following after induction (optionally Dox induction), fluorescent (optionally E2-Crimson negative cells) are sorted out;
(c) the enzyme is supplied in trans in an independent inducible vector and the Gal4-substrate fusion is supplied or expressed constitutively; or
(d) the enzyme is supplied in cis as part of a Gal4-substrate fusion or as part of a Gal4 fusion construct.

24. (canceled)

25: The isolated, synthetic or recombinant polypeptide or peptide of claim 1, wherein: the polypeptide or peptide of (a), or the peptidomimetic or bioisostere of (b), further comprises, or is modified by: acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation and/or arginylation


SEQUENCE LISTING (12 sequences)

SEQ ID NO: 1 - protein, 9 aa, artificial sequence
  synthetic peptide
TRRVAHNSE

SEQ ID NO: 2 - protein, 9 aa, artificial sequence
  synthetic peptide
WRGAAMVRG

SEQ ID NO: 3 - protein, 8 aa, artificial sequence
  synthetic peptide
RVVAHNSD

SEQ ID NO: 4 - protein, 7 aa, artificial sequence
  synthetic peptide
TRVVAHN

SEQ ID NO: 5 - protein, 9 aa, artificial sequence
  synthetic peptide
TKVVAHNSD

SEQ ID NO: 6 - protein, 8 aa, artificial sequence
  artificial peptide
RVVAQNSE

SEQ ID NO: 7 - protein, 9 aa, artificial sequence
  artificial peptide
TRVIAHTSE

SEQ ID NO: 8 - protein, 8 aa, artificial sequence
  artificial peptide
WRGAAMAR

SEQ ID NO: 9 - protein, 10 aa, artificial sequence
  artificial peptide
WKDRGAAMVR

SEQ ID NO: 10 - protein, 10 aa, artificial sequence
  artificial peptide
WRGAALCMVR

SEQ ID NO: 11 - protein, 10 aa, artificial sequence
  artificial peptide
WRGAVCAMVR

SEQ ID NO: 12 - protein, 10 aa, artificial sequence
  artificial peptide
WRGRAAMVKG